(19) United States
(12) Patent Application Publication
(10) Pub. No.: US 2023/0134432 A1
(43) Pub. Date: May 4, 2023

(54) COMPOSITIONS AND METHODS FOR TREATING SOLID AND SOFT TUMORS AND PROLIFERATIVE DISEASES

(71) Applicant: NOVEL CONCEPTS MEDICAL LTD, Tel Aviv (IL)

(72) Inventor: Rachel ALKALAY, Tel Aviv (IL)

(73) Assignee: NOVEL CONCEPTS MEDICAL LTD, Tel Aviv (IL)

(21) Appl. No.: 17/912,779

(22) PCT Filed: Mar. 19, 2021
(86) PCT No.: PCT/IL2021/050310

§ 371 Date: Sep. 19, 2022

Related U.S. Application Data
(60) Provisional application No. 62/992,276, filed on Mar. 20, 2020.

(51) Int. Cl. A61K 36/53; A61K 36/185; A61K 36/22; A61K 36/424; A61K 36/324; A61K 36/258; A61K 36/71; A61K 31/122; A61K 31/05; A61K 38/48; A61K 36/88; A61K 31/405; A61K 36/534; A61K 36/738; A61K 36/899; A61K 36/85; A61K 36/9062; A61K 36/54; A61K 36/82; A61P 35/00

(52) U.S. Cl. CPC A61K 36/53; A61K 36/185; A61K 36/22; A61K 36/424; A61K 36/324; A61K 36/258; A61K 36/71; A61K 31/122; A61K 31/05; A61K 38/4873; A61K 36/88; A61K 31/405; A61K 36/534; A61K 36/738; A61K 36/899; A61K 36/85; A61K 36/9062; A61K 36/54; A61K 36/82; A61P 35/00

(57) ABSTRACT

A method of preventing or treating solid and soft tumors and proliferative diseases in a subject in need thereof is provided. The method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein the component is capable of treating solid and soft tumors and proliferative diseases and wherein the plant species is selected from the group consisting of Nigella sativa, Thymuscapitatus, Thymus vulgaris, Origanum syriacum, Thymbra spicata, Satujera thymbra, Sesamum indicum, Rhus coriaria, Gynostemma pentaphyllum, Boswellia sacra and Panaxginseng, preventing or treating solid and soft tumors and proliferative diseases in the subject.

### FIELD AND BACKGROUND OF THE INVENTION

The present invention, in some embodiments thereof, relates to compositions and methods for treating solid and soft tumors and proliferative diseases.

The word “cancer” is used to describe a number of diseases in which there is uncontrolled division of abnormal cells. Cancerous solid and soft tumors and proliferative diseases may initially arise in virtually any tissue or organ in the body and forms as a result of a complex interaction of both innate genetic factors and environmental factors, such as one's diet or exposure to radiation, toxins, and the like. Despite advances in medicine and the understanding of the molecular basis of cancerous solid and soft tumors and proliferative diseases, the exact causes of any given type of cancer are largely unknown, especially in a particular individual. Given this lack of knowledge, it is not surprising that it remains highly difficult to find effective treatments for solid and soft tumors and proliferative diseases.

Finding effective treatments is also made challenging because cancer often develops resistance to various therapeutic strategies. In addition, effective means for treating cancer become an even greater challenge in view of the capacity for certain types of cancers to spread from their primary source. This process, called metastasis, enables tumor cells to spread to other vital parts of the body through the blood and lymph systems. Metastasis is a key reason why effective cancer treatments are difficult to develop.

Existing cancer therapies today include multiple different ablation techniques such as surgical procedures; cryogenic or heat methods on the tissue, ultrasound, radiofrequency, and radiation; chemical methods such as pharmaceuticals, cytotoxic agents, monoclonal antibodies; or transarterial chemo immobilization (TACE), and combinations thereof pursuant to specific regimens based on the specific type and stage of cancer under treatment. However, these therapies are associated with substantially high costs. In addition, current treatment options are highly invasive, are associated with significant toxicities, and result in an overall poor quality of life for patients.

Standard of care cancer tumor therapies typically couple surgical removal of the affected tissue with chemotherapy or radiation treatments. Standard approaches for administering chemotherapeutics are through the blood, e.g., systemic delivery, which can be achieved by various routes such as intravenous and/or gastrointestinal delivery. However, toxicity is a major drawback associated with systemically delivered chemotherapeutic drugs. Standard of care surgical treatments also introduce problems, including dislodgement of cancer cells into the blood and/or lymph systems, which results in the opportunity for cancer cells to metastasize to other sites in the body and cause additional tumors to form.

When surgery is not possible, the accepted treatment for solid and soft tumors and proliferative diseases is to use radiation or chemotherapy. But survival rates for inoperable cancer are low when compared to the survival rate for tumors that are surgically removed prior to chemotherapy or radiation.

Certain cancer tumors resemble the body's tissue and thus diminish the immune system's otherwise innate ability to identify and kill them. Several cancer-fighting technologies (e.g., cancer vaccines) aim to stimulate the immune system against cancerous cells. The immune system's ability to mount an attack on the tumor cell is hindered because the tumor cell displays few, if any, antigens that are foreign to that individual. In addition, a tumor can have many different types of cells in it. Each cell type has different cell-surface antigens, again thwarting attack by the immune system.

Depending on disease stage, the tumor may be too advanced (e.g., bulky) for the vaccine to be effective. These, as well as other factors, are why tumors may lack sufficient amounts of antigens (or targets) needed to stimulate a sufficient immune system.

It general, if cancer is detected early, the standard treatments against cancer can be highly effective. However, even when the best results are obtained, such treatments are invasive, toxic and damaging to the body and mentally and emotionally demanding on the patient. If cancer is detected in late stage, few treatments offer the patient much hope of long term survival.

Thus, there continues to be a need in the art to identify and develop new strategies that are more effective at treating solid and soft tumors and proliferative diseases, and which present lower costs to individuals and society in general.

### SUMMARY OF THE INVENTION

According to an aspect of the invention there is provided a method of preventing or treating a solid and soft tumors and proliferative diseases in a subject in need thereof, the method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein the component is capable of treating the solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng, preventing or treating solid and soft tumors and proliferative diseases in the subject.

According to an aspect of the invention there is provided a vaccine against a solid and soft tumors and proliferative diseases comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of treating or preventing solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

According to an aspect of the invention there is provided a pharmaceutical composition comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of treating or preventing solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng for use in preventing or treating solid and soft tumors and proliferative diseases.

According to an aspect of the invention there is provided a composition of matter comprising at least 2 of a plant species or genus thereof-derived components selected from the group consisting of a plant part, extract thereof, fraction thereof, active ingredient thereof, synthetic analog thereof, mimetic thereof or combination thereof, wherein the component is capable of treating solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

According to an aspect of the invention there is provided a food supplement comprising a combination of at least 2 of a plant species or genus thereof-derived component selected from the group consisting of a plant part, extract thereof, fraction thereof, active ingredient thereof, synthetic analog thereof, mimetic thereof or combination thereof, wherein the component is capable of treating solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum and Panax ginseng Gynostemma pentaphyllum, Boswellia sacra and Panax ginseng.

According to an aspect of the present invention the method, vaccine, pharmaceutical composition, composition or food supplement comprises bromelain or an analog thereof. According to an aspect of the present invention the method, vaccine, pharmaceutical composition, composition or food supplement comprises extracts of pineapple comprising bromelain or an analog thereof.

According to an aspect of the present invention the method, vaccine, pharmaceutical composition, composition or food supplement comprises extracts of plants containing Tryptophan.

According to an aspect of the invention there is provided a food supplement, composition or extracts further including “Beduin Tea” comprising

Rose Leaves Micromeria fruticose, Salvia, cymbopgon (Citral,) Aloysia, verbena officinalis, Origanum majorana, menthe

According to an aspect of the invention there is provided a food supplement, composition or extracts further including “Beduin Tea” comprising
Thyme, sage, cardamom, cinnamon, black tea, habuk, Marmaya.
Further details of components of Thyme Vulgaris are included in APPENDIX1.

According to some embodiments of the invention the solid and soft tumors and proliferative diseases is selected from the group consisting of sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioblastoma multiforme, astrocytoma, medulloblastoma, craniopharyngioma, ependymoma, pinealoma, hemangioblastoma, acoustic neuroma, oligodendroglioma, cutaneous T cell lymphoma (CTCL), melanoma, neuroblastoma, and retinoblastoma.

According to some embodiments of the invention, the solid and soft tumors and proliferative diseases is brain cancer, breast cancer, triple negative breast cancer, bladder cancer, bone cancer, colorectal cancer, lung cancer, kidney cancer, liver cancer, stomach cancer, prostate cancer, sarcoma, melanoma, carcinoma, or a lymphoma.

According to specific embodiments of the invention, the solid and soft tumors and proliferative diseases is prostate cancer, breast cancer, colorectal cancer, pancreatic cancer, or a lymphoma.

According to some embodiments of the invention the solid and soft tumors and proliferative diseases is a lymphoma.

According to some embodiments of the invention the proliferative disease is Fibroids

According to some embodiments of the invention the proliferative disease is Endometriosis

According to some embodiments of the invention, the component comprises at least 2 components.

According to some embodiments of the invention, the component comprises at least 3 components.

According to some embodiments of the invention, the component comprises at least 4 components.

According to some embodiments of the invention, the component comprises at least 5 components.

According to some embodiments of the invention, the component comprises 5-10 components.

According to some embodiments of the invention, the component comprises thymoquinone or an analog thereof.

According to some embodiments of the invention, the component comprises thymol or an analog thereof.

According to some embodiments of the invention, the component comprises carvacrol or an analog thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C shows embodiments in plant extraction methods as taken from berkem(dot)com. FIG. 1A—scheme describing the general principle of plant extraction; FIG. 1B—scheme describing the main separation process according to some embodiments; FIG. 1C—scheme describing parameters that may influence the process.

FIG. 2 depict the discoloration and pigmentation of a patient with Squamous Cell Carcinoma suspected of a Basal Cell Carcinoma involvement before treatment, dated Jul. 28, 2020.

FIG. 3 depict the discoloration and pigmentation of a patient with Squamous Cell Carcinoma suspected of a Basal Cell Carcinoma involvement after treatment, dated Oct. 30, 2020.

### DESCRIPTION OF SPECIFIC EMBODIMENTS OF THE INVENTION

The present invention, in some embodiments thereof, relates to compositions and methods for treating and preventing solid and soft tumors and proliferative diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The target tissue of this invention is solid and soft tumors and proliferative diseases, and in particular, malignant solid and soft tumors and proliferative diseases. Provided herein are plant-based compositions and/or components for treating solid and soft tumors and proliferative diseases. A solid and soft tumors and proliferative diseases refers to an abnormal mass of tissue that usually does not contain cysts or liquid areas. The plant-based compositions or components of the invention, when administered to a subject suffering from the solid and soft tumors and proliferative diseases can have a therapeutic effect (to name a few, the compositions and/or components can alleviate symptoms of the solid and soft tumors and proliferative diseases, cause regression of the tumor mass, slow the progress of the cancer or cure the cancer). At this time, there are no effective preventative treatments for many of the forms of solid and soft tumors and proliferative diseases and accompanying symptoms thereof.

Thus, according to an aspect of the invention there is provided a method of treating a solid and soft tumors and proliferative diseases in a subject in need thereof, the method comprising administering to the subject an effective amount of a plant species or genus thereof-derived component selected from the group consisting of a plant part, extract thereof, fraction thereof, active ingredient thereof, synthetic analog thereof, mimetic thereof or combination thereof, wherein the component is capable of treating solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng, preventing or treating solid and soft tumors and proliferative diseases in the subject.

According to an alternative or an additional aspect of the invention there is provided a vaccine against solid and soft tumors and proliferative diseases growth comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of treating and/or slowing the growth of solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

According to an alternative or an additional aspect of the invention there is provided a pharmaceutical composition comprising an effective amount of a plant species or genus thereof-derived component selected from the group consisting of a plant part, extract thereof, fraction thereof, active ingredient thereof, synthetic analog thereof, mimetic thereof or combination thereof, wherein the component is capable of treating solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng for use in treating solid and soft tumors and proliferative diseases.

According to an alternative or an additional aspect of the invention there is provided a composition of matter comprising at least 2 of a plant species or genus thereof—derived components selected from the group consisting of a plant part, extract thereof, fraction thereof, active ingredient thereof, synthetic analog thereof, mimetic thereof or combination thereof, wherein the component is capable of treating solid and soft tumors and proliferative diseases and wherein the plant species is selected from the group consisting of Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

According to embodiments of the present invention the method, vaccine, pharmaceutical composition, composition or food supplement comprises bromelain or an analog thereof. According to embodiments of the present invention the method, vaccine, pharmaceutical composition, composition or food supplement comprises extracts of pineapple comprising bromelain or an analog thereof.

It is herein acknowledged that aspects of the present invention provide compositions and methods for the treatment or prevention of Oncoviruses.

### Oncoviruses:

An oncovirus or oncogenic virus is a virus that can cause cancer. Oncovirus herein refers to any virus with a DNA or RNA genome causing cancer and is synonymous with “tumor virus” or “cancer virus”.
Kaposi's sarcoma is a cancer that can form masses in the skin and is caused by the Kaposi's sarcoma-associated herpesvirus (KSHV), also called HHV-8.
DNA virusesHuman papillomavirus (HPV), a DNA virus, causes transformation in cells through interfering with tumor suppressor proteins such as p53. Interfering with the action of p53 allows a cell infected with the virus to move into a different stage of the cell cycle, enabling the virus genome to be replicated. Forcing the cell into the S phase of the cell cycle could cause the cell to become transformed.^{[25]} Human papillomavirus infection is a major cause of cervical cancer, vulvar cancer, vaginal cancer, penis cancer, anal cancer, and HPV-positive oropharyngeal cancers.^{[7][26][27][28][30][31]} There are nearly 200 distinct human papillomaviruses (HPVs),^{[29]} and many HPV types are carcinogenic.^{[7][26]}
Kaposi's sarcoma-associated herpesvirus (KSHV or HHV-8) is associated with Kaposi's sarcoma, a type of skin cancer)³²
Epstein-Barr virus (EBV or HHV-4) is associated with four types of cancers
Merkel cell polyomavirus—a polyoma virus—is associated with the development of Merkel cell carcinoma^{[24]}
Human cytomegalovirus (CMV or HHV-5) is associated with mucoepidermoid carcinoma and possibly other malignancies.^{[33]}

### RNA Viruses

Some RNA viruses have also been associated with cancer such as the hepatitis C virus as well as certain retroviruses, e.g., human T-lymphotropic virus (HTLV-1) and Rous sarcoma virus (RSV).

The term ‘“plant” as used herein encompasses whole plants, a grafted plant, ancestors and progeny of the plants and plant parts, including seeds, flowers, bark, shoots, stems, roots (including tubers), fruit, rootstock, scion, and plant cells, tissues and organs.

According to a specific embodiment, the plant part is a seed.

According to a specific embodiment, the plant part is a fruit.

According to a specific embodiment, the plant part is a leaf.

According to a specific embodiment, the plant part is a stem.

According to a specific embodiment, the plant part is a flower.

The plant part can be a solid part or a non-solid part such as oil or aqueous portions of the plant.

The plant may be in any form including suspension cultures, embryos, meristematic regions, callus tissue, leaves, gametophytes, sporophytes, pollen, and microspores.

The term plant refers to a wild plant or a cultivated variety thereof.

As used herein the term “plant species” refers to a sub-group of one or more plants within the genus. These plants will share similar characteristics with each other. There may be a single plant within a species, or there may be many hundreds of plants. The term intends to include subspecies, such as grown or can be found in different geographical location, e.g., Lebanese Sumac and Syrian Sumac or Korean Ginseng and American Ginseng.

As used herein “plant genus” refers to a taxonomic rank below family and above species.

It will be appreciated that the relevant species and genera and listed below and each option or combination thereof represents a different embodiment of the invention.

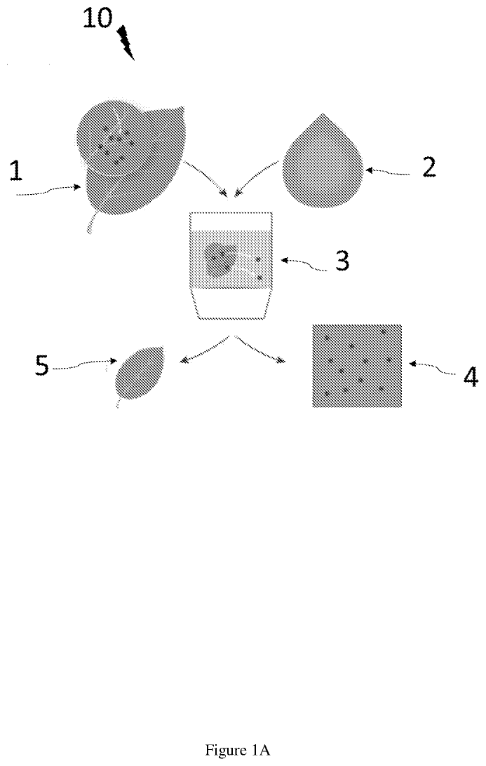

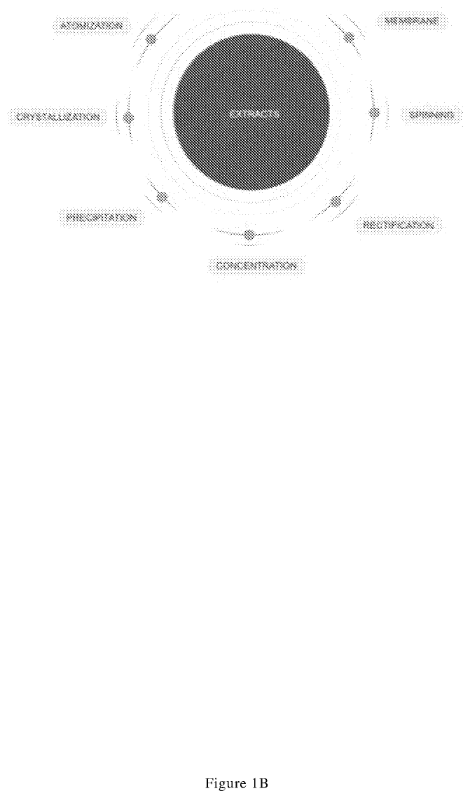

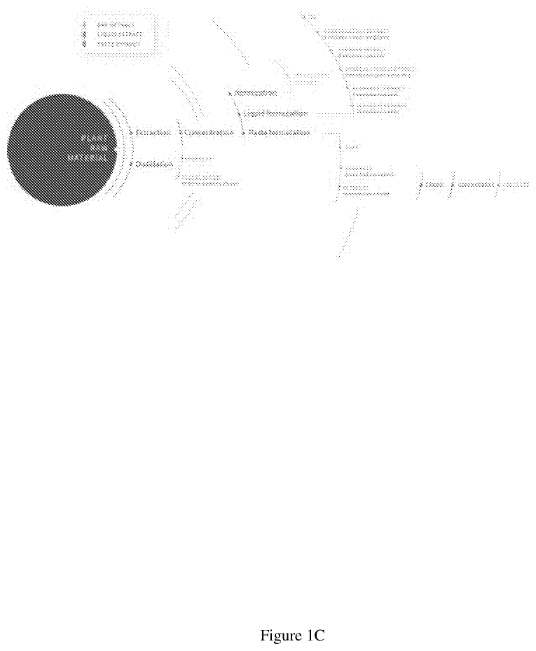

The term ‘extraction” refers to a separation process which relies on the separation of one or more analytes from the components of a sample other than the one or more analytes. Extractions are processes that typically use two immiscible phases to separate one or more solutes from one phase into the other. The distribution of a solute between two phases is an equilibrium condition described by partition theory. For example, boiling tea leaves in water extracts the tannins, theobromine, and caffeine out of the leaves and into the water. More typical extractions preformed typically but not only in a laboratory are settings of organic compounds out of an aqueous phase and into an organic phase. Common extractants are arranged from ethyl acetate to water (ethyl acetate<acetone<ethanol<methanol<acetone: water (7:3)<ethanol:water (8:2)<methanol:water (8:2)<water) in increasing order of polarity according to the Hildebrand solubility parameter. Procedures for plant extraction are provided in FIGS. 1A-C.

The term “extract” as used herein refers to the result of such process of separation that can take the form of a solution formulation or other chemical form depending on the extraction process. In particular, the term extract can relate to a substance made by extracting a part of a sample (e.g. a raw material), such as by using a solvent such as ethanol or water. In various instances an extract relates to a solvent that is enriched in one or more solute. In particular, a “plant extract” in the sense of the present disclosure typically comprises a concentrated preparation of a plant material obtained by isolating or purifying desired active constituents with one or more extraction processes.

The choice of the solvent depends on the desired component to be obtained. For example, to extract polar components in an extraction process suggested solvents include, but are not limited to, water, ethanol methanol or butanol while for non polar compounds diethyl ether, hexane or chloroform depending on the use of the extract. For midpolar one may choose Ethyl acetate but other solvants can be used as well.

The general procedure of solid/liquid extraction can be scaled in five different ways:

Maceration: the contact stage is maintained at room temperature.

Decoction or reflux: the contact stage is maintained at the boiling point of the solvent.

Digestion: the contact stage is maintained at a temperature in between those of the previous two cases.

Infusion: the boiling solvent is poured over the solid, then left to cool for a set time.

Leaching or percolation: the solvent passes through the biomass.

It is also possible to combine these methods with each other or with other processes such as distillation, steam distillation, rectification, etc.

According to another embodiment, the use of various solvents, either successively or in combination is contemplated and the ordinary skilled of organic chemistry will know which to choose according to the active ingredient as described below.

Extraction may be further assisted by other means such as ultrafiltration, reverse osmosis, high pressure (supercritical CO2), microwaves, ultrasound, etc.

In some embodiments, the plant part is contacted with a polar solvent (e.g. ethanol) or nonpolar solvent (e.g., hexane or pentane) for several minutes, e.g., 15 minutes or more, about 30 minutes or more, about 1 hour or more, about 2 hours or more, or about 5 hours or more.

Temperature can also be controlled during the contacting.

According to specific embodiments, the plant part is contacted with the solvent (e.g. ethanol) while being constantly mixed e.g. on a shaker.

It will be appreciated that the extraction process can also be solvent-free.

For example, solvent-free microwave extraction (SFME) has been proposed as a green method for the extraction of essential oil from aromatic herbs that are extensively used in the food industry. This technique is a combination of microwave heating and dry distillation performed at atmospheric pressure without any added solvent or water. The isolation and concentration of volatile compounds is performed in a single stage. In some embodiments, SFME and/or hydro-distillation (HD), are used for the extraction of essential oil from the plants of the invention.

In some embodiments, the process of the present invention comprises isolating a liquid extract (i.e. filtered extract) from the mixture (i.e. crude extract) comprising the liquid extract and solids. Suitable means for isolating the liquid extract (i.e. filtered extract) include those known in the art of organic synthesis and include, but are not limited to, gravity filtration, suction and/or vacuum filtration, centrifuging, setting and decanting, and the like. In some embodiments, the isolating comprises filtering a liquid extract through a porous membrane, syringe, sponge, zeolite, paper, or the like having a pore size of about 1-5 μm, about 0.5-5 μm, about 0.1-5 μm, about 1-2 μm, about 0.5-2 μm, about 0.1-2 μm, about 0.5-1 μm, about 0.1-1 μm, about 0.25-0.45 μm, or about 0.1-0.5 μm (e.g. about 2 μm, about 1 μm, about 0.45 μm, or about 0.25 μm).

According specific embodiments, the present invention contemplates drying (i.e. removal of the polar/non-polar solvent) and/or freezing the filtered extract following generation thereof.

The method for drying the filtered extract (i.e. removing the polar solvent) is not particularly limited, and can include solvent evaporation at a reduced pressure (e.g., sub-atmospheric pressure) and/or an elevated temperature (e.g., above about 25° C.). In some embodiments, it can be difficult to completely remove a solvent from a liquid extract by standard solvent removal procedures such as evaporation. In some embodiments, processes such as co-evaporation, lyophilization, and the like can be used to completely remove the polar solvent from a liquid fraction to form a dry powder, dry pellet, dry granulate, paste, and the like. According to a specific embodiment the polar solvent is evaporated with a vacuum evaporator.

The selection of the extraction process much depends on the component to be isolated.

It will be appreciated that following generation of the extract, specific embodiments of the present invention further contemplate additional purification steps so as to further isolate/purify active agents from the extract, for example, by fractionating the filtered extract.

As used herein “a fraction” refers to a portion of the extract that contains only certain chemical ingredients of the extract but not all.

Fractionating can be performed by processes such as, but not limited to: column chromatography, preparative high performance liquid chromatography (“HPLC”), reduced pressure distillation, and combinations thereof.

According to a specific embodiment, fractionating is performed by HPLC.

In some embodiments, fractionating comprises re-suspending the filtered extract in a polar solvent (such as methanol, as discussed above), applying the polar extract to a separation column, and isolating the extract having the anti-respiratory disease (e.g. anti-fibrotic, anti-inflammatory) activity by column chromatography (preparative HPLC).

An eluting solvent is applied to the separation column with the polar extract to elute fractions from the polar extract. Suitable eluting solvents for use include, but are not limited to, methanol, ethanol, propanol, acetone, acetic acid, carbon dioxide, methylethyl ketone, acetonitrile, butyronitrile, carbon dioxide, ethyl acetate, tetrahydrofuran, di-iso-propylether, ammonia, triethylamine, N,N-dimethylformamide, N,N-dimethylacetamide, and the like, and combinations thereof.

According to an alternative or an additional embodiment, liquid chromatography comprises high performance liquid chromatography (HPLC).

According to an alternative or an additional embodiment, liquid chromatography is performed on a reverse stationary phase.

The fractions may be characterized by analytical methods such as, but not limited to, spectroscopic methods such as, but not limited to, ultraviolet-visible spectroscopy (“UV-Vis”), infrared spectroscopy (“IR”), and the like; mass-spectrometry (“MS”) methods such as, but not limited to, time-of-flight MS; quadrupole MS; electrospray MS, Fourier-transform MS, Matrix-Assisted Laser Desorption/Ionization (“MALDI”), and the like; chromatographic methods such as, but not limited to, gas-chromatography (“GC”), liquid chromatograph (“LC”), high-performance liquid chromatography (“HPLC”), and the like; and combinations thereof (e.g., GC/MS, LC/MS, HPLC/UV-Vis, and the like), and other analytical methods known to persons of ordinary skill in the art.

The component (active ingredients, extract and/or fractions) obtained may be tested for treating solid and soft tumors and proliferative diseases or symptoms thereof. Exemplary methods for testing the effect are further described herein below as well as in the Examples section which follows.

The active ingredients, extract and/or fraction described herein may be immediately used or stored until further used.

According to specific embodiments, the active ingredients, extract and/or fractions is kept frozen, e.g. in a freezer, until further use (e.g. at about −20° C. to −90° C., at about −70° C. to −90° C., e.g. at −80° C.), for any required length of time.

According to other specific embodiments, the active ingredients, extract and/or fractions is immediately used (e.g. within a few minutes e.g., up to 30 minutes).

The active ingredients, extract and/or fractions may be used separately. Alternatively, different active ingredients, extract and/or fractions (e.g. from different plants or from separate extraction procedures) may be pooled together. Likewise, different active ingredients, extract and/or fractions (from the same extract, from different extracts, from different plants and/or from separate extraction procedures) may be pooled together.

Using the present teachings, the present inventor was able to identify not only plants and extracts that can be used to effectively treat or prevent solid and soft tumors and proliferative diseases, but also active ingredients thereof.

“Active ingredient” refers to a defined chemical composition which is responsible for the anti (preventive or therapeutic) effect against solid and soft tumors and proliferative diseases.

The active ingredient can be purified from a plant or chemically synthesized (artificial, man-made).

Also contemplated herein are analogs and derivatives of the active ingredients as long as the anti-tumor (preventive or therapeutic) effect against solid and soft tumors and proliferative diseases is maintained (see e.g., Examples section which follows), which are also referred to as mimetics.

Following are some non-limiting examples for extraction of active ingredients from selected plants of the present invention.

Extraction from leaves of T. capitatus—The Aerial parts of T. capitatus (leaves) samples are collected. Leaves separated from branches are dehydrated at room temperature for 7 days and slightly blended into fine powders for extractions.

Essential oil (EO) extraction—hydro-distillation is used to extract EO from the plant, e.g., dried aerial parts of T. capitatus. In brief, the extraction is conducted for several hours for example, 3 h, by mixing 100 g of plants in 500 mL of distilled water. The extract is dried and concentrated using sodium sulphate and rotatory evaporator under reduced pressure. The EO yield is established by quantity of the obtained oil in mL for 100 g of dried plant. Finally, the pure EO os stored at −4° C. until further analyzed.

Essential oil analysis—The chemical composition of EO is examined by GC and GC-MS. GC analysis is conducted using gas chromatograph. The proportion of the constituents is determined by the integration of peak areas. In addition, mass spectrometry (MS) can be used to analyze the EO typically under the same conditions as described above for gas chromatography. The identification of the different compounds is defined by comparison of their retention indexes (determined relatively to the retention times of a series of n-alkanes) with those of standards of the Wiley library search routines12, based on fit and purity of mass spectra. Such conditions are used for determining the active ingredients as described below.

Extraction from Satuiera Thymbra:

Air dried aerial parts from S. thymbra were collected in Lebanon at random during April 2009. For 3 h the plant material was submitted to steam distillation using a clevenger-type apparatus to produce the essential oil with a yield of 0.84% (w/w). Oil is dried using anhydrous magnesium sulfate and stored at 4° C. S. thymbra oil was analyzed by GC/MS.

Extraction from Rhus coriaria (Sumac)

In order to isolate, determine and identify the compounds from the Rhus coriaria fruits, different extracts are taken from the fruit or leaves of the Sumac plant. Some are isolated from aqueous extracts, others from alcoholic extracts and some from lipid extracts. Hydrolysable tannins compose the highest percentage in the Sumac fruits, followed by flavonoids. This emphasizes the antioxidant potential of the fruit. Following hydrolysable tannins, comprising almost 20% of the fruit's mass, are other unidentified compounds. Subsequently there are anthocyanins, isoflavonoids, terpenoids and diterpenes. Analysis of the chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Extraction of Thymoquinone from Nigella sativa

Various methods can be used including microwave-assisted extraction system having temperature controlling function as well as other extraction methods, Soxhlet and conventional solid/liquid extraction.

### Nigella Sativa

| 24-ETHYL-LOPHENOL | Seed Oil | | |
|---|---|---|---|
| 24-METHYL-LOPHENOL | Seed Oil | | |
| 24-METHYLENE-CYCLOARTANOL | Seed Oil | | |
| 5-DEHYDRO-AVENASTEROL | Seed Oil | | |
| 7-DEHYDRO-AVENASTEROL | Seed Oil | | |
| ALANINE | Seed | 8,000 | 10,255 |
| ALPHA-SPINASTEROL | Seed Oil | | |
| | Seed | | |
| ARABIC-ACID | Seed | | |
| ARACHIDIC-ACID | Seed Oil | | 1,900 |
| ARACHIDONIC-ACID | Seed Oil | | 24,900 |
| ARGININE | Seed | 41,500 | 53,050 |
| ASCORBIC-ACID | Leaf | | 2,577 |
| ASH | Seed | 38,000 | 53,000 |
| ASPARAGINE | Seed | | 0 |
| ASPARTIC-ACID | Seed | 10,670 | 13,650 |
| ASTRAGALIN | Seed | | 200 |
| BETA-AMYRIN | Seed Oil | | |
| BETA-SITOSTEROL | Seed Oil | | |
| | Seed | | 3,218 |
| BUTYROSPERMOL | Seed Oil | | |
| CALCIUM | Seed | | 10,600 |
| CAMPESTANOL | Seed Oil | | |
| CAMPESTEROL | Seed Oil | | |
| | Seed | | |
| CARBOHYDRATES | Seed | | 339,600 |
| CARVONE | Seed | | |
| | Essent. Oil | | |
| | Seed | 2,250 | 9,600 |
| CHOLESTEROL | Seed Oil | | |
| | Seed | | |
| CITROSTADIENOL | Seed Oil | | |
| CYCLOARTENOL | Seed Oil | | |
| CYCLOEUCALENOL | Seed Oil | | |
| CYMENE | Seed | | |
| CYSTINE | Seed | | |
| D-LIMONENE | Seed | | |
| DAMASCENINE | Plant | | |
| DEHYDROASCORBIC-ACID | Leaf | | 295 |
| DITHYMOQUINONE | Seed Oil | | |
| EICOSADIENOIC-ACID | Seed Oil | | 25,000 |
| | Seed | 8,979 | 10,525 |
| EO | Seed Oil | | 4,500 |
| | Seed | 4,000 | 16,000 |
| FAT | Seed | 354,900 | 416,000 |
| FIBER | Seed | | 55,000 |
| FIXED-OIL | Seed | | 380,000 |
| GLUCOSE | Seed | | |
| GLUTAMIC-ACID | Seed | 28,085 | 35,900 |
| GLYCINE | Seed | 8,840 | 20,700 |
| GRAMISTEROL | Seed Oil | | |
| HEDERAGENIN | Seed | | 10,000 |
| INDOLE-3-ACETIC-ACID | Tissue | | 14 |
| | Culture | | |
| IRON | Seed | | 140 |
| ISOLEUCINE | Seed | 8,570 | 10,960 |
| KAEMPFEROL-3-O-BETA-GLUCOPYRANOSYL-(1,2)-O-BETA- | Seed | | 100 |
| GALACTOPYRANOSYL-(1,2)-BETA-GLUCOPYRANOSIDE | | | |
| LEUCINE | Seed | 23,130 | 29,595 |
| LINOLEIC-ACID | Seed Oil | 487,600 | 576,300 |
| | Seed | 128,124 | 233,459 |
| LINOLENIC-ACID | Seed Oil | 5,700 | 7,000 |
| | Seed | 2,484 | 2,912 |
| LIPASE | Seed | | |
| LOPHENOL | Seed Oil | | |
| LYSINE | Seed | 16,200 | 20,700 |
| MELANTHIGENIN | Seed | | |
| MELANTHIN | Plant | | 15,000 |
| | Seed | | 15,000 |
| METHIONINE | Seed | 13,100 | 16,750 |
| MYRISTIC-ACID | Seed Oil | 1,900 | 18,000 |
| | Seed | 567 | 1,082 |
| NIGELLICINE | Seed | | |
| NIGELLIDINE | Seed | | |
| NIGELLIMINE | Seed | | 0.13 |
| NIGELLIMINE-N-OXIDE | Seed | | 0.2 |
| NIGELLIN | Seed | | |
| NIGELLINE | Seed | | |
| | Essent. Oil | | |
| NIGELLONE | Seed | | |
| | Essent. Oil | | |
| | Plant | | |
| | Seed | | |
| OBTUSIFOLIOL | Seed Oil | | |
| OLEIC-ACID | Seed Oil | 244,600 | 262,400 |
| | Seed | 89,911 | 184,912 |
| PALMITIC-ACID | Seed Oil | 120,000 | 171,200 |
| | Seed | 22,464 | 50,523 |
| PALMITOLEIC-ACID | Seed Oil | | 2,000 |
| PHENYLALANINE | Seed | 16,850 | 21,560 |
| PHYTOSTEROLS | Seed | | 5,100 |
| POTASSIUM | Seed | | 5,820 |
| PROLINE | Seed | 11,350 | 14,520 |
| PROTEIN | Seed | 210,000 | 271,900 |
| QUERCETIN-3′-GLUCOSIDE | Seed | | 440 |
| QUERCETIN-3-O-(6-FERULOYL-BETA-GLUCOPYRANOSYL)-(1,2)- | Seed | | 240 |
| BETA-GALACTOPYRANOSYL-(1,2)-BETA-GLUCOPYRANOSIDE | | | |
| QUERCETIN-3-O-BETA-GLUCOPYRANOSYL-(1,2)-O-BETA- | Seed | | 1,380 |
| GALACTOPYRANOSYL-(1,2)-BETA-GLUCOPYRANOSIDE | | | |
| RESIN | Seed | | |
| RUTIN | Seed | | 200 |
| SERINE | Seed | 4,210 | 5,385 |
| SODIUM | Seed | | 980 |
| STEARIC-ACID | Seed Oil | 18,100 | 60,400 |
| | Seed | 8,722 | 10,192 |
| STIGMAST-7-EN-3-BETA-OL | Seed Oil | | |
| STIGMASTANOL | Seed Oil | | |
| STIGMASTEROL | Seed Oil | | |
| | Seed | | |
| TANNIN | Seed | | |
| TARAXEROL | Seed Oil | | |
| TELFAIRIC-ACID | Seed Oil | | |
| THREONINE | Seed | 2,615 | 3,345 |
| THYMOHYDROQUINONE | Seed | | |
| THYMOL | Seed Oil | | |
| THYMOQUINONE | Seed | | |
| | Essent. Oil | | |
| | Seed Oil | | |
| | Seed | | |
| TIRUCALLOL | Seed Oil | | |
| TRYPTOPHAN | Seed | | |
| TYROSINE | Seed | 12,925 | 16,530 |
| VALINE | Seed | 6,500 | 8,325 |

According to a specific embodiment, active ingredients (e.g., which can be obtained by supercritical carbon dioxide extraction method) include but are not limited to:

| Compound | RIₑₓₚ | RIₗᵢₜ | SFE 1 | SFE 2 | HD SFE | Identification |
|---|---|---|---|---|---|---|
| n-Nonane^{a} | 905 | 900 | 0.12 | — | — | RI, MS |
| Tricyclene | 926 | 926 | tr | — | — | RI, MS |
| Camphene | 953 | 953 | — | — | 1.64 | RI, MS |
| β-Pinene | 958 | 959 | — | — | 0.40 | RI, MS |
| 2,4,(10)-Thujadiene | 967 | 960 | 4.74 | 0.19 | — | RI, MS |
| Sabinene | 978 | 977 | 1.05 | — | — | RI, MS |
| β-Myrcene | 990 | 991 | 0.31 | — | — | RI, MS |
| 1,8-Cineole | 1013 | 1010 | — | — | 0.98 | RI, MS |
| α-Terpinene | 1025 | 1026 | 2.34 | — | — | RI, MS |
| Limonene | 1034 | 1034 | 0.18 | 0.38 | 1.03 | RI, MS |
| γ-Terpinene | 1054 | 1056 | 27.46 | 13.20 | 12.87 | RI, MS |
| cis-Sabinene hydrate | 1063 | 1068 | — | 0.38 | Tr | RI, MS |
| allo-Ocimenol^{a} | 1079 | 1071 | — | 0.11 | — | RI, MS |
| Linalool | 1087 | 1080 | 0.25 | 0.19 | — | RI, MS |
| Terpinolene | 1091 | 1088 | — | — | Tr | RI, MS |
| trans-Sabinene hydrate | 1099 | 1097 | 0.37 | — | — | RI, MS |
| Terpinen-1-ol^{a} | 1124 | 1120 | — | — | 0.11 | RI, MS |
| 1,5,8-p-Menthatriene^{a} | 1130 | 1135 | 0.43 | 0.38 | | RI, MS |
| Borneol | 1152 | 1152 | — | — | 1.02 | RI, MS |
| Pinocarvone | 1167 | 1165 | 2.96 | 3.00 | — | RI, MS |
| trans-Dihydrocarvone | 1208 | 1202 | — | 0.19 | — | RI, MS |
| Dihydrocarvone^{a} | 1215 | 1214 | 0.37 | 2.06 | — | RI, MS |
| Ocimenone (E)^{a} | 1249 | 1239 | 1.54 | 1.50 | — | RI, MS |
| Thymoquinone | 1250 | 1250 | 35.05 | 33.12 | 38.41 | RI, MS, NMR |
| Thymol | 1283 | 1288 | 7.43 | 5.30 | 16.95 | RI, MS, NMR |
| Carvacrol | 1299 | 1299 | 1.98 | 1.73 | 0.81 | RI, MS |
| 2-Undecanone | 1312 | 1315 | — | — | 13.72 | RI, MS |
| n-Octyl isobutyrate^{a} | 1323 | 1326 | — | — | 0.12 | RI, MS |
| α-Longipinene | 1330 | 1334 | 0.26 | — | — | RI, MS |
| Citronellyl acetate^{a} | 1339 | 1336 | — | — | 0.50 | RI, MS |
| Thymohydroquinone methyl ether^{a} | 1353 | 1351 | — | — | Tr | RI, MS |
| Cyclosativene | 1367 | 1366 | — | — | 1.43 | RI, MS |
| α-Longicyclene | 1381 | 1380 | 0.43 | 5.25 | — | RI, MS |
| α-Copaene | 1385 | 1383 | 1.54 | 2.00 | 0.41 | RI, MS |
| α-Longifolene | 1391 | 1387 | — | — | 0.51 | RI, MS |
| (Z)-Caryophyllene^{a} | 1395 | 1395 | 0.23 | — | — | RI, MS |
| β-Caryophyllene | 1420 | 1417 | 2.89 | 5.07 | 4.80 | RI, MS |
| Thymohydroquinone dimethylether^{a} | 1429 | 1425 | 0.43 | — | — | RI, MS |
| Aromadendrene^{a} | 1437 | 1438 | — | — | 1.04 | RI, MS |
| Thymohydroquinone | 1515 | 1509 | 1.17 | 1.12 | 2.31 | RL, MS, NMR |
| Davanone^{a} | 1587 | 1586 | 0.31 | — | — | RI, MS |
| 8-Heptadecene^{a} | 1683 | 1680 | 1.23 | 1.13 | 0.86 | RI, MS |
| Dihydrofarnesyl acetate^{a} | 1841 | 1840 | 2.28 | 4.69 | — | RI, MS |
| Pimaradiene^{a} | 1934 | 1935 | 1.23 | 2.25 | — | RI, MS |
| Palmitic acid | 1947 | 1946 | 0.18 | — | — | RI, MS |
| Pimara-8(14), 15-diene | 1968 | 1966 | 0.92 | — | — | RI, MS |
| Octadecanoic acid | 2145 | 2157 | 0.26 | 12.31 | — | RI, MS |
| Total identified | | | 99.94 | 95.55 | 99.92 | |
| Grouped compounds: | | | | | | |
| Quinones | | | 44.08 | 39.54 | 57.67 | |
| Monoterpene hydrocarbons | | | 36.51 | 14.15 | 15.94 | |
| Oxygenated monoterpenes | | | 7.47 | 9.16 | 17.14 | |
| Sesquiterpene hydrocarbons | | | 5.35 | 12.32 | 8.19 | |
| Oxygenated sesquiterpenes | | | 2.59 | 4.69 | — | |
| Diterpenes | | | 2.15 | 2.25 | — | |
| Alkane | | | 0.12 | — | — | |
| Alkenes | | | 1.23 | 1.13 | 0.86 | |
| Fatty acids | | | 0.44 | 12.31 | — | |
| Fatty acid esters | | | | — | 0.12 | |

Additional plants that are contemplated herein are of the genus Nigella.

Nigella is a genus of 18 species of annual plants in the family Ranunculaceae, native to Southern Europe, North Africa, South Asia, Southwest Asia and Middle East. Common names applied to members of this genus are nigella, devil-in-a-bush or love-in-a-mist.

Nigella arvensis
Nigella carpatha
Nigella damascena
Nigella degenii
Nigella deserti
Nigella doerfleri
Nigella elata
Nigella fumariifola
Nigella hispanica
Nigella latisecta
Nigella nigellastrum
Nigella orientalis
Nigella oxypetala
thymo
Nigella sativa
Nigella segetalis
Nigella stricta
Nigella unguicularis

According to a specific embodiment the active ingredient is thymoquinone.

Additional plants containing thymoquinone include, but are not limited to:

Monarda fistulos (of the genus Monarda);
Satureja montana (of the genus Satujera);

Additional families containing thymoquinone include, but are not limited to: Asteraceae—examples include, but are not limited to the subfamilies:
Barnadesioideae Bremer & Jansen
Carduoideae Sweet
Cichorioideae Chevallier
Corymbioideae Panero & Funk
to Famatinanthoideae S. E. Freire, Ariza & Panero
Gochnatioideae Panero & Funk
Gymnarrhenoideae Panero & Funk
Hecastocleidoideae Panero & Funk
Mutisioideae Lindley
Pertyoideae Panero & Funk
Stifftioideae Panero
Wunderlichioideae Panero & Funk

### Cupressaceae

Cunninghamioideae
Taiwanioideae
Athrotaxidoideae
Sequoioideae
Taxodioideae
Callitroideae
Cupressoideae
Incertae sedis

### Lamiacea

### Ranunculacea

Hydrastidoideae
Glaucidioideae
Coptoideae
Thalictroideae
Ranunculoideae

List of plants that contain Carvacrol include, but are not limited to:

Monarda didyma
Nigella sativa
Origanum compactum
Origanum dictamnus
Origanum microphyllum
Origanum onites
Origanum scabrum
Origanum syriacum
Origanum vulgare
Plectranthus amboinicus
Thymus glandulosus
Lavandula multifida
Origanum minutiflorum
Satureja thymbra
Active Ingredients Found in Thymus capitatus

| No | RI | Compound | % |
|---|---|---|---|
| 1 | 935 | α-Thujene | 0.54 |
| 2 | 940 | α-Pinene | 0.38 |
| 3 | 991 | Myrcene | 0.87 |
| 4 | 1019 | α-terpinene | 1.11 |
| 5 | 1025 | p-Cymene | 6.25 |
| 6 | 1063 | γ-Terpinene | 6.75 |
| 7 | 1089 | α-terpinolene | 0.26 |
| 8 | 1101 | Linalool | 1.51 |
| 9 | 1179 | Terpinen-4-ol | 1.40 |
| 10 | 1185 | 4-Carvomenthenol | 0.94 |
| 11 | 1260 | Geraniol | 0.25 |
| 12 | 1309 | Carvacrol | 65.38 |
| 13 | 1310 | Thymol | 1.35 |
| 14 | 1358 | Eugenol | 0.21 |
| 15 | 1408 | Carvacryl Acetate | 0.45 |
| 16 | 1427 | β-Caryophyllene | 4.94 |
| 17 | 1461 | α-Humulene | 0.10 |
| 18 | 1487 | allo-aromadendrene | 0.18 |
| 19 | 1685 | α-Bisabolol | 0.35 |
| 20 | 1774 | α-Bisabolol oxide A | 0.11 |
| 21 | 1815 | Hexadecanal | 0.14 |
| 22 | 1870 | 1-Hexadecanol | 0.46 |
| 23 | 1879 | 1-Hexadecanol | 0.13 |
| 24 | 1894 | Rimuene | 0.28 |
| 25 | 1957 | Hexadecanoic acid | 0.68 |
| | | Total identified | 95.02 |
| | | Unknown | 4.98 |

Additional plants contemplated herein are of the genus Thymus.

The genus Thymus (/’-tarm [symbol] s/TY-m [symbol] s; thymes) contains about 350 species of aromatic perennial herbaceous plants and subshrubs to 40 cm tall in the family Lamiaceae, native to temperate regions in Europe, North Africa and Asia.

Stems tend to be narrow or even wiry; leaves are evergreen in most species, arranged in opposite pairs, oval, entire, and small, 4-20 mm long, and usually aromatic. Thyme flowers are in dense terminal heads with an uneven calyx, with the upper lip three-lobed, and are yellow, white, or purple.

Several members of the genus are cultivated as culinary herbs or ornamentals, when they are also called thyme after its best-known species, Thymus vulgaris or common thyme.

About 350 species, including:

Thymus adamovicii
Thymus altaicus
Thymus amurensis
Thymus boissieri
Thymus bracteosus
Thymus broussonetii
Thymus caespititius
Thymus camphoratus
Thymus capitatus
Thymus capitellatus
Thymus camphoratus
Thymus carnosus
Thymus cephalotus
Thymus cherlerioides
Thymus ciliatus
Thymus cilicicus
Thymus cimicinus
Thymus citriodorus (Thymus×citriodorus) syn. T. fragrantissimus, T. serpyllum citratus, T. serpyllum citriodorum.^{[7]}—citrus thyme
Thymus comosus
Thymus comptus
Thymus curtus
Thymus decussatus
Thymus disjunctus
Thymus doerfleri
Thymus glabrescens
Thymus herba-barona
Thymus hirsutus
Thymus hyemalis
Thymus inaequalis
Thymus integer
Thymus lanuginosus, syn. T. serpyllum—woolly thyme
Thymus leucospermus
Thymus leucotrichus
Thymus longicaulis
Thymus longiflorus
Thymus mandschuricus
Thymus marschallianus
Thymus mastichina
Thymus membranaceus
Thymus mongolicus
Thymus moroderi
Thymus nervulosus
Thymus nummularis
Thymus odoratissimus
Thymus pallasianus
Thymus pallidus
Thymus pannonicus
Thymus praecox—creeping thyme
Thymus proximus
Thymus pseudolanuginosus, syn. T. serpyllum—woolly thyme
Thymus pulegioides—lemon thyme^{[8]}
Thymus quinquecostatus
Thymus richardii
Thymus satureioides
Thymus serpyllum
Thymus sibthorpii
Thymus striatus
Thymus thracicus—lavender thyme
Thymus villosus
Thymus vulgaris—common thyme
Thymus zygis

List of plants that contain thymol include, but are not limited to:

Euphrasia rostkoviana
Lagoecia cuminoides
Monarda didyma
Monarda fistulosa
Mosla chinensis, Xiang Ru
Origanum compactum
Origanum dictamnus
Origanum onites
Origanum vulgare
Satureja thymbra
Thymus glandulosus
Thymus hyemalis
Thymus vulgaris
Thymus zygis
Trachyspermum ammi

Active ingredients in Thymus vulgaris:

| | | | |
|---|---|---|---|
| | Plant | Low | High |
| Chemical | part | ppm | ppm |

| | | |
|---|---|---|
| 1-OCTEN-3-OL | Shoot | 150 |
| | Shoot | 65 |
| | Shoot | 80 |
| 2,5-DIETHYL-TETRAHYDROFURAN | Shoot | 0 |
| | Shoot | 6 |
| | Shoot | 6 |
| 3-OCTANOL | Shoot | 110 |
| | Shoot | 12 |
| | Shoot | 30 |
| ALPHA-GUAIENE | Shoot | 0.1 |
| | Shoot | 6 |
| | Shoot | 0 |
| ALPHA-HUMULENE | Shoot | 45 |
| | Shoot | 20 |
| | Shoot | 55 |
| ALPHA-PHELLANDRENE | Shoot | 0 |
| | Shoot | 40 |
| | Shoot | 12 |
| ALPHA-PINENE | Shoot | 0 |
| | Shoot | 265 |
| | Shoot | 325 |
| ALPHA-TERPINENE | Shoot | 840 |
| | Shoot | 990 |
| | Shoot | 990 |
| ALPHA-TERPINEOL | Shoot | 55 |
| | Shoot | 55 |
| | Shoot | 25 |
| ALPHA-THUJENE | Shoot | 320 |
| | Shoot | 0 |
| | Shoot | 0 |
| BETA-CARYOPHYLLENE | Shoot | 175 |
| | Shoot | 185 |
| | Shoot | 200 |
| BETA-GUAIENE | Shoot | 0.1 |
| | Shoot | 0 |
| | Shoot | 3 |
| BETA-PHELLANDRENE | Shoot | 80 |
| | Shoot | 60 |
| BETA-PHELLLANDRENE | Shoot | 70 |
| BETA-PINENE | Shoot | 30 |
| | Shoot | 30 |
| | Shoot | 560 |
| BORNEOL | Shoot | 55 |
| | Shoot | 30 |
| | Shoot | 15 |
| CAMPHENE | Shoot | 30 |
| | Shoot | 25 |
| | Shoot | 40 |
| CAMPHOR | Shoot | 0 |
| | Shoot | 0.1 |
| | Shoot | 0 |
| CARVACROL | Shoot | 1,285 |
| | Shoot | 24,850 |
| | Shoot | 23,765 |
| CARVONE | Shoot | 15 |
| | Shoot | 20 |
| | Shoot | 0.1 |
| CARYOPHYLLENE-OXIDE | Shoot | 75 |
| | Shoot | 55 |
| | Shoot | 45 |
| CIS-CARVEOL | Shoot | 0 |
| | Shoot | 0 |
| | Shoot | 3 |
| CIS-SABINENE-HYDRATE | Shoot | 20 |
| | Shoot | 0 |
| | Shoot | 55 |
| CITRONELLOL | Shoot | 12 |
| | Shoot | 0.1 |
| | Shoot | 0 |
| CITRONELLOL-BUTYRATE | Shoot | 0 |
| CITRONELLYL-BUTYRATE | Shoot | 0 |
| | Shoot | 15 |
| DIHYDROCARVONE | Shoot | 0 |
| | Shoot | 0 |
| | Shoot | 12 |
| EHTYL-CINNAMATE | Shoot | 0 |
| EO | Shoot | 31,000 |
| | Shoot | 31,000 |
| | Shoot | 31,000 |
| ETHYL-CINNAMATE | Shoot | 0 |
| | Shoot | 30 |
| GAMMA-TERPINENE | Shoot | 2,700 |
| | Shoot | 1,015 |
| | Shoot | 240 |
| GERANIOL | Shoot | 0 |
| | Shoot | 0 |
| | Shoot | 65 |
| GERANYL-ACETATE | Shoot | 0 |
| | Shoot | 0 |
| | Shoot | 15 |
| GERANYL-BUTYRATE | Shoot | 0 |
| | Shoot | 20 |
| | Shoot | 0 |
| GERANYL-HEXANOATE | Shoot | 0 |
| | Shoot | 0 |
| | Shoot | 6 |
| GERANYL-PROPIONATE | Shoot | 0 |
| | Shoot | 0 |
| | Shoot | 70 |
| GERMACRENE-D | Shoot | 0 |
| | Shoot | 0 |
| | Shoot | 50 |
| LIMONENE | Shoot | 110 |
| | Shoot | 55 |
| | Shoot | 90 |
| LINALOL | Shoot | 35 |
| | Shoot | 55 |
| | Shoot | 25 |
| METHYL-2-METHYL-BUTYRATE | Shoot | 6 |
| | Shoot | 12 |
| | Shoot | 9 |
| MYRCENE | Shoot | 750 |
| | Shoot | 565 |
| | Shoot | 0.1 |
| P-CYMENE | Shoot | 4,445 |
| | Shoot | 1,880 |
| | Shoot | 3,135 |
| TERPINEN-1-OL | Shoot | 6 |
| | Shoot | 15 |
| | Shoot | 0 |
| TERPINEN-4-OL | Shoot | 435 |
| | Shoot | 315 |
| | Shoot | 335 |
| TERPINOLENE | Shoot | 0 |
| | Shoot | 0.1 |
| | Shoot | 45 |
| THYMOL | Shoot | 18,560 |
| | Shoot | 385 |
| | Shoot | 280 |
| TRANS-BERGAMOTENE | Shoot | 9 |
| | Shoot | 9 |
| TRANS-BERGAMOTTENE | Shoot | 9 |
| TRANS-SABINENE-HYDRATE | Shoot | 25 |
| | Shoot | 120 |
| | Shoot | 0 |
| TRICYCLENE | Shoot | 0 |
| | Shoot | 0 |
| | Shoot | 3 |

Active ingredients on the EO of Thymus vulgaris according to some embodiments of the invention, include, but are not limited to:

| No. | RT (min) | Area % of total | Constituents* |
|---|---|---|---|
| 1 | 5.39 | 1.06 | alpha-Thujene |
| 2 | 5.63 | 1.07 | alpha-Pinene |
| 3 | 6.89 | 0.37 | beta-Pinene |
| 4 | 6.97 | 1.53 | beta-Myrcene |
| 5 | 7.53 | 0.33 | alpha-Phellandrene |
| 6 | 7.77 | 3.76 | Carene<δ-2-> |
| 7 | 8.04 | 0.29 | D-Limonene |
| 8 | 8.26 | 0.21 | beta-Phellandrene |
| 9 | 8.46 | 8.41 | para-Cymene |
| 10 | 8.96 | 30.90 | gamma-Terpinene |
| 11 | 9.48 | 0.47 | Terpineol |
| 12 | 12.55 | 0.46 | Terpinen-4-ol |
| 13 | 16.17 | 47.59 | Thymol |
| 14 | 17.32 | 2.68 | Caryophyllene |
| 15 | 19.03 | 0.78 | Cyclohexene, 1-methyl-4-(5- |
| | | | methyl-1-methylene-4-hexenyl) |
| Total | | 99.91% | |

Active Ingredients of Satujera Thymbra:

Air dried aerial parts from S. thymbra were collected in Lebanon at random during April 2009. For 3 h the plant material was submitted to steam distillation using a clevenger-type apparatus to produce the essential oil with a yield of 0.84% (w/w). Oil was dried using anhydrous magnesium sulfate and stored at 4° C. S. thymbra oil are analyzed by GC/MS. Nineteen compounds representing 98.8% of the oil sample are identified. The major components of Satureja thymbra L. oil are γ-terpinene (34.06%), carvacrol (23.07%) and thymol (18.82%). Also abundant are ρ-cymene (7.58%), caryophyllene (3.96%), α-terpinene (3.53%) and myrcene (1.70%).

Also contemplated herein are plants of the genus Satujera. Satureja is a genus of aromatic plants of the family Lamiaceae, related to rosemary and thyme. It is native to North Africa, southern and southeastern Europe, the Middle East, and Central Asia. A few New World species were formerly included in Satureja, but they have all been moved to other genera. Several species are cultivated as culinary herbs called savory, and they have become established in the wild in a few places.

Examples include, but are not limited to:

Satureja adamovicii Šilic—Balkans
Satureja aintabensis P. H. Davis—Turkey
Satureja amani P. H. Davis—Turkey
Satureja atropatana Bunge—Iran
Satureja avromanica Maroofi—Iran
Satureja bachtiarica Bunge—Iran
Satureja boissieri Hausskn. ex Boiss.—Turkey, Iran
Satureja bzybica Woronow—Caucasus
Satureja×caroli-paui G. López—Spain (S. innota×S. montana)
Satureja cilicica P. H. Davis—Turkey
Satureja coerulea Janka—Bulgaria, Romania, Turkey
Satureja cuneifolia Ten—Spain, Italy, Greece, Albania, Yugoslavia, Iraq
Satureja×delpozoi Sánchez-Gómez, J. F. Jiménez & R. Morales—Spain (S. cuneifolia×S. intricata var. gracilis)
Satureja edmondii Briq.—Iran
Satureja×exspectata G. López—Spain (S. intricata var. gracilis×S. montana)
Satureja fukarekii Šilk—Yugoslavia
Satureja hellenica Halácsy—Greece
Satureja hortensis L.
Satureja horvatii Šilk—Greece, Yugoslavia
Satureja icarica P. H. Davis—Greek Islands
Satureja innota (Pau) Font Quer—Spain
Satureja intermedia C. A. Mey.—Iran, Caucasus
Satureja intricata Lange—Spain
Satureja isophylla Rech.f.—Iran
Satureja kallarica Jamzad—Iran
Satureja kermanshahensis Jamzad—Iran
Satureja khuzistanica Jamzad—Iran
Satureja kitaibelii Wierzb. ex Heuff.—Bulgaria, Romania, Yugoslavia
Satureja laxiflora K. Koch—Iran, Iraq, Turkey, Caucasus
Satureja linearifolia (Brullo & Furnari) Greuter—Cyrenaica region of Libya
Satureja macrantha C. A. Mey.—Iran, Iraq, Turkey, Caucasus
Satureja metastasiantha Rech.f.—Iraq
Satureja montana L.—winter savory—southern Europe, Turkey, Syria
Satureja mutica Fisch. & C. A. Mey.—Caucasus, Iran, Turkmenistan
Satureja nabateorum Danin & Hedge—Jordan
Satureja×orjenii Šilic—Yugoslavia (S. horvatii×S. montana)
Satureja pallaryi J. Thiébaut—Syria
Satureja parnassica Heldr. & Sart. ex Boiss.—Greece, Turkey
Satureja pilosa Velen.—Italy, Greece, Bulgaria
Satureja rumelica″ Velen.—Bulgaria
Satureja sahendica Bornm.—Iran
Satureja salzmannii (Kuntze) P. W. Ball—Morocco, Spain
Satureja spicigera (K. Koch) Boiss.—Turkey, Iran, Caucasus
Satureja spinosa L.—Turkey, Greek Islands including Crete
Satureja subspicata Bartl. ex Vis.—Austria, Yugoslavia, Albania, Bulgaria, Italy
Satureja taurica Velen.—Crimea
Satureja thymbra L.—Libya, southeastern Europe from Sardinia to Turkey; Cyprus, Lebanon, Palestine
Satureja thymbrifolia Hedge & Feinbrun—Israel, Saudi Arabia
Satureja visianii Šilic.—Yugoslavia
Satureja wiedemanniana (Avé-Lall.) Velen.—Turkey

Active ingredients of Thymbra spicata:

| Compounds | % | RI¹ | RT² |
|---|---|---|---|
| α-pinene | 0.56 | 1028 | 3.64 |
| α-phellandrene | 0.64 | 1033 | 3.71 |
| camphene | 0.06 | 1073 | 4.36 |
| β-pinene | 0.10 | 1113 | 5.16 |
| δ-3-carene | 0.05 | 1155 | 6.10 |
| β-myrcene | 1.04 | 1170 | 6.51 |
| α-terpinene | 1.48 | 1184 | 6.90 |
| dl-limonene | 0.17 | 1202 | 7.43 |
| β-phellandrene | 0.12 | 1212 | 7.69 |
| γ-derpinene | 10.73 | 1252 | 8.86 |
| p-cymene | 12.18 | 1276 | 9.69 |
| α-terpinolene | 0.05 | 1286 | 10.04 |
| oct-1-en-3-ol | 0.11 | 1454 | 16.17 |
| trans sabinene hydrate | 0.05 | 1465 | 16.59 |
| cis sabinene hydrate | 0.03 | 1547 | 19.73 |
| linalool | 0.03 | 1551 | 19.91 |
| trans caryophyllene | 1.28 | 1589 | 21.39 |
| 4-terpineol | 0.53 | 1598 | 21.79 |
| isoborneol | 0.21 | 1694 | 25.36 |
| d-carvone | 0.02 | 1728 | 26.55 |
| anethole | 0.04 | 1826 | 30.05 |
| caryophyllene oxide | 0.65 | 1968 | 34.87 |
| spathulenol | 0.15 | 2125 | 39.56 |
| thymol | 2.77 | 2218 | 41.80 |
| carvacrol | 66.86 | 2239 | 42.61 |
| naphthalene³ | 0.08 | 2281 | 44.26 |

¹RT—retention time;
²RI—retention index;
³naphthalene,1,2,3,4,4a,5,6,7-octahydro-4a-methyl

Also contemplated herein are plants of the genus Thymbra.

Thymbra, common name Mediterranean thyme, is a genus of plants in the family Lamiaceae. As currently categorized, the genus has seven species and one subspecies. It is native to the Mediterranean region of southern Europe, North Africa, and the Middle East.

Examples include, but are not limited to:

Thymbra calostachya (Rech.f.) Rech.f.—Crete
Thymbra capitata (L.) Cay.—widespread from Morocco+Portugal to Turkey+Palestine
Thymbra sintenisii Bornm. & Am.—Iraq, Turkey
Thymbra spicata L.—Greece, Turkey, Syria, Lebanon, Palestine, Israel, Iraq, Iran
Thymbra thymbrifolia (Hedge & Feinbrun) Brauchler, comb. nov.—Israel, Palestine, Judean Desert, Khirbet el Mird
Thymbra nabateorum (Danin & Hedge) Brauchler, comb. nov.—W of Jordan and the adjacent N of Saudi Arabia
Thymbra linearifolia (Brullo & Furnari) Brauchler, comb. nov.—Libya

Chemical Composition of Rhus coriaria (Sumac)

Characterization and identification of chemical compounds of Sumac using HPLC-MS method identified 191 compounds in Rhus coriaria and classified them as generally being:
78 hydrolysable tannins (e.g., gallotannins, e.g., penta, hexa, hepta, octa, nona and decagalloyl-glucoside)
59 flavonoids (e.g., Quercetin, Myrecetin 3-rhamnoside and Quercetin 3-glucoside)
9 anthocyanins (e.g., Delphidin-3-glucoside, Cyanidin 3-(2″-galloyl)galactoside, Cyanidin-3-glucoside, 7-methyl-cyanidin-3-(2″galloyl)galactoside, 7-methyl-cyanidin-3-galactoside)
2 isoflavonoids
2 terpenoids
1 diterpene
38 other unidentified compounds.

According to specific embodiments, the phenolic compounds in Sumac are the compounds that constitute its phytochemical activity along with anthocyanins. The most abundant phenolic compound in sumac fruits was found to be Gallic acid.

Hydrolysable tannins compose the highest percentage in the Sumac fruits, followed by flavonoids. This emphasizes the antioxidant potential of the fruit, a plant part contemplated herein as a specific embodiment. Following hydrolysable tannins, comprising almost 20% of the fruit's mass, are other unidentified compounds. Subsequently there are anthocyanins, isoflavonoids, terpenoids and diterpenes. The chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Hydrolysable tannins compose the highest percentage in the Sumac fruits, followed by flavonoids. This emphasizes the antioxidant potential of the fruit. Following hydrolysable tannins, comprising almost 20% of the fruit's mass, are other unidentified compounds. Subsequently there are anthocyanins, isoflavonoids, terpenoids and diterpenes. The chemical properties of sumac fruit is conducted on ripe fruits and have found a 2.6% protein content, 7.4% fat content, 14.6% fiber content, 1.8% ash. Also, a calorimetric calculation showed that 100 g of sumac fruit contains 147.8 kcal.

Other active ingredients or any combinations thereof include, but are not limited to, methyla gallate, gathisflavone, sumaflavone, hinfikflavone, photocatechuic acid, penta-galloylglucose, hinokiflavone, β-caryophyllene, Delphidin-3-glucoside, Cyanidin 3-(2″-galloyl)galactoside, Cyanidin-3-glucoside, 7-methyl-cyanidin-3-(2″galloyl)galactoside, 7-methyl-cyanidin-3-galactoside, quercetin-3-glucoside, kampferol, myricetin, butein, D-limonine.

According to a specific embodiment, the active ingredient or combination thereof includes a volatile compound, e.g., terpene hydrocarbons, monoterpene and sesquiterpene hydrocarbons, specifically β-caryophyllene and α-pinene, Coririanaphthyl ether, Coriarioic acid and Coriariacthracenyl ester.

According to a specific embodiment, the active ingredient or combination thereof includes a fatty acid, e.g., oleic acid, linoleic acid, palmitic acid, β-caryophillene, cembrene stearic acid, Myristic acid, α-linolenic acid.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., thiamin B₁, riboflavin B₂, pyridoxine B₆, cyanocobalamin B₁₂, nicotinamide, biotin and ascorbic acid.

According to a specific embodiment, a methanol or ethanol extract is performed, e.g., ethanol concentration is 80%; extraction time is 1 h; extraction temperature is 40° C.; particle size 1.0 mm; and solvent to sumac ratios 15:1 ml/g. Other extraction procedures include, but are not limited to, those described in Sakhr and Khatib Heliyon. 2020 January; 6(1): e03207, which is hereby incorporated by reference in its entirety.

According to another embodiment, the plant part is leaf.

Also contemplated herein are plants of the genus Rhus.

Examples include, but are not limited to:

Asia and southern Europe
Rhus chinensis Mill.—Chinese sumac
Rhus coriaria—Tanner's sumac
Rhus delavayi Franchet

### Australia, Pacific

Rhus taitensis Guill. (Northeast Australia, Malesia, Micronesia, French Polynesia)
Rhus sandwicensis A. Gray—neneleau (Hawaii)

### North America

Rhus aromatica—fragrant sumac
Rhus copallinum—winged or shining sumac
Rhus glabra—smooth sumac
Rhus integrifolia—lemonade sumac
Rhus kearneyi—Kearney sumac
Rhus lanceolata—prairie sumac
†Rhus malloryi Wolfe & Wehr—Ypresian, Washington
Rhus michauxii—Michaux's sumac
Rhus microphylla—desert sumac, littleleaf sumac
Rhus ovata—sugar sumac
†Rhus republicensis Flynn, DeVore, & Pigg-Ypresian, Washington
†Rhus rooseae Manchester—Middle Eocene, Oreg.
Rhus trilobata Nutt.—skunkbush sumac
Rhus typhina—staghom sumac
Rhus virens Lindh. ex A. Gray— evergreen sumac

Chemical Composition of Panax ginseng (Ginseng)

Characterization and identification of chemical compounds of Ginseng using a variety of methods identified a large variety of compounds in Panax ginseng and classified them as generally being:
Saponin Glycosides (e.g., ginsenosides)
Phytosterols (e.g. stigmasterol, beta-sterol)
Sesquiterpenes (e.g. beta-alamene and beta-selinine)
Flavenoids (e.g. Kaempferol)
Polyacetylenes (e.g. panaxynol, ginsenoyne A)
Alkaloids (e.g. fumarine, girinimbin)
Polysaccharides
Phenolic compounds (e.g. elemicin, dauricin, maltol).

According to specific embodiments, the saponin compounds in Ginseng and the polysaccharide compounds are the compounds that constitute its phytochemical activity. The most abundant saponin compound in ginseng root was found to be ginsenoside. Polysaccharides from ginseng have been identified as NGP, WGP, 1-KGP, 4-KGP, WGPE and EGP, with WGP and WGPE being the most abundant, depending on the species of ginseng plant material used for extraction.

Most ginseng saponins belong to a family of steroids with a four trans-ring rigid steroid skeleton. They are also referred to as ginsenosides, triterpenoid saponins or dammarane derivatives. More than 200 saponins have been isolated from ginseng plants.

In addition to ginseng root, saponins have been identified in ginseng leaves and stems, flower buds, fruits, berries, and seeds. Because steaming or heating changes the saponin profile of ginseng products, ginseng saponins have also been identified in the processed root, leaf, flower-bud and berry.

Ginseng saponins are divided into several groups. Two major groups are the protopanaxadiol (PPD)-type saponins with sugar moieties attached to the C-3 and/or C-20 and the protopanaxatriol (PPT) group with sugar moieties at C-6 and/or at C-20. Other groups include the ocotillol-type with a five-membered epoxy ring at C-20, the oleanane-type with a nonsteroidal structure, and the dammarane type with a modified C-20 side chain. As techniques are developed for chemical purification and structural identification, novel ginseng saponins continue to be discovered.

The table below shows ginsenoside compounds recovered from ginseng extracts prepared by different extraction procedures:

Ginsenosides

| | Solvent system^{a} | | | |
|---|---|---|---|---|
| Material | (volume ratio) | Detection^{b} | Obtained compound | Isolation efficiency^{c} |
| P. | Hex-n-BuOH—H₂O | TLC | Ginsenosides Rb1, | 157, 13, 56, and 17 mg of |
| notoginseng, | (3:4:7) | | Re, Rg1 and | Rb1, Re, Rg1 and R1 from |
| root | | | notoginsenoside R1 | 283 mg MeOH extract of |
| | | | | five tablets |
| P. ginseng, | CH₂Cl₂—MeOH—NH₄OAc—iPrOH | ELSD | Ginsenosides Rf, Rd, | 10.7, 11.0, 13.4 and 13.9 |
| root | (6:2:4:3) | | Re, and Rb1 | mg of Rf, Rd, Re and Rb1 |
| | | | | from 480 mg enriched |
| | | | | fraction by macroporous |
| | | | | resin |
| P. | CHCl₃—MeOH-2-BuOH—H₂O | ELSD | Ginsenosides Rg1, | Not provided |
| notoginseng, | (5:6:1:4) | | Rd, Re, Rb1 and | |
| root | EtOAc—n-BuOH—H₂O | | notoginsenoside R1 | |
| | (1:1:2) | | | |
| Red P. | CH₂Cl₂—MeOH—H₂O—iPrOH | ELSD | Ginsenosides Rg3, | 32.2, 26.6, 28.6 and 8.1 |
| ginseng, | (6:6:4:1) | | Rk1, Rg5 and F4 | mg of Rg3, Rk1, Rg5 and |
| steamed root | | | | F4 from 350 mg enriched |
| | | | | fraction by RP-C₁₈ column |
| P. ginseng, | EtOAc—iPrOH-0.1% | UV | Ginsenoside Ro | 61 mg Ro from 100 mg |
| root | formic acid | | | enriched sample by |
| | H₂O (3:1:5) | | | normal-phase MPLC |

^{a}Abbreviations: Hex: n-hexane; BuOH: butanol; CH₂Cl₂: methylene chloride; MeOH: methanol; NH₄OAc: ammonium acetate; iPrOH: isopropanol; CHCl₃: chloroform; EtOAc: ethyl acetate.
^{b}Abbreviations: TLC: thin layer chromatography; ELSD: evaporative light scattering detection; UV: ultraviolet.
^{c}Abbreviations: RP: reversed-phase; MPLC: medium-pressure liquid chromatography.

The table below shows the chemical formulae of 123 dammarane-type saponins isolated from various parts of Panax plants. They are placed in the order of the structure type.

Dammarane—Type Saponin Ginsenosides

| No. | Name | Formula | Plant Material |
|---|---|---|---|
| 1 | Floralginsenoside M | C₅₃H₉₀O₂₂ | Flower buds of P. ginseng |
| 2 | Floralginsenoside N | C₅₃H₉₀O₂₂ | Flower buds of P. ginseng |
| 3 | Floralquinquenoside E | C₅₃H₉₀O₂₂ | Flower buds of P. quinquefolius |
| 4 | Ginsenoside Rh5 | C₃₇H₆₄O₉ | Roots and rhizomes of P. vietnamensis |
| 5 | Notoginsenoside FP1 | C₄₇H₈₀O₁₈ | Fruit pedicels of P. notoginseng |
| 6 | Notoginsenoside M | C₄₈H₈₂O₁₉ | Roots of P. notoginseng |
| 7 | Notoginsenoside N | C₄₈H₈₂O₁₉ | Roots of P. notoginseng |
| 8 | Notoginsenoside Rw1 | C₄₆H₇₈O₁₇ | Rhizomes of P. notoginseng |
| 9 | Notoginsenoside T3 | C₃₈H₆₆O₉ | Acid hydrolysate roots of P. |
| | | | notoginseng |
| 10 | Notoginsenoside U | C₄₂H₇₂O₁₄ | Roots of P. notoginseng |
| 11 | Quinquenoside L17 | C₄₇H₈₀O₁₈ | Leaves and stems of P. quinquefolius |
| 12 | Yesanchinoside D | C₄₄H₇₄O₁₅ | Underground part of P. japonicus |
| 13 | Yesanchinoside E | C₅₄H₉₂O₂₃ | Underground part of P. japonicus |
| 14 | Yesanchinoside F | C₅₆H₉₄O₂₄ | Underground part of P. japonicus |
| 15 | 20(S)-acetylated Rg2 | C₄₄H₇₄O₁₄ | Roots of P. quinquefolius |
| 16 | 20(R)-acetylated Rg2 | C₄₄H₇₄O₁₄ | Roots of P. quinquefolius |
| 17 | Malonylginsenoside Ra3 | C₆₂H₁₀₂O₃₀ | Fresh roots of P. ginseng |
| 18 | Malonylnotoginsenoside R4 | C₆₂H₁₀₂O₃₀ | Roots of P. ginseng |
| 19 | Notoginsenoside FP2 | C₅₈H₉₈O₂₆ | Fruit pedicels of P. notoginseng |
| 20 | Notoginsenoside FT1 | C₄₇H₈₀O₁₇ | Acid hydrolysate roots of P. |
| | | | notoginseng |
| 21 | Notoginsenoside L | C₅₃H₉₀O₂₂ | Roots of P. notoginseng |
| 22 | Notoginsenoside O | C₅₂H₈₈O₂₁ | Flower buds of P. notoginseng |
| 23 | Notoginsenoside P | C₅₂H₈₈O₂₁ | Flower buds of P. notoginseng |
| 24 | Notoginsenoside Q | C₆₃H₁₀₆O₃₀ | Flower buds of P. notoginseng |
| 25 | Notoginsenoside S | C₆₃H₁₀₆O₃₀ | Flower buds of P. notoginseng |
| 26 | Notoginsenoside T | C₆₄H₁₀₈O₃₁ | Flower buds of P. notoginseng |
| 27 | Quinquenoside L10 | C₄₇H₈₀O₁₇ | Leaves and stems of P. quinquefolius |
| 28 | Quinquenoside L14 | C₄₇H₈₀O₁₇ | Leaves and stems of P. quinquefolius |
| 29 | Yesanchinoside J | C₆₁H₁₀₂O₂₈ | Underground part of P. japonicus |
| 30 | Floralginsenoside A | C₄₂H₇₂O₁₆ | Flower buds of P. ginseng |
| 31 | Floralginsenoside C | C₄₁H₇₀O₁₅ | Flower buds of P. ginseng |
| 32 | Floralginsenoside H | C₅₀H₈₄O₂₁ | Flower buds of P. ginseng |
| 33 | Floralginsenoside J | C₄₈H₈₂O₂₀ | Flower buds of P. ginseng |
| 34 | Floralginsenoside Ka | C₃₆H₆₂O₁₁ | Flower buds of P. ginseng |
| 35 | Floralginsenoside Tc | C₅₃H₉₀O₂₄ | Flower buds of P. ginseng |
| 36 | Floralquinquenoside B | C₄₂H₇₂O₁₅ | Flower buds of P. quinquefolius |
| 37 | Floralquinquenoside D | C₄₂H₇₂O₁₅ | Flower buds of P. quinquefolius |
| 38 | Floranotoginsenoside B | C₅₃H₉₀O₂₄ | Flowers of P. notoginseng |
| 39 | Floranotoginsenoside C | C₅₃H₉₀O₂₄ | Flowers of P. notoginseng |
| 40 | Ginsenoside I | C₄₈H₈₂O₂₀ | Flower buds of P. ginseng |
| 41 | Ginsenoside II | C₄₈H₈₂O₂₀ | Flower buds of P. ginseng |
| 42 | Ginsenoside SL1 | C₃₆H₆₂O₁₁ | Steamed leaves of P. ginseng |
| 43 | Floralginsenoside B | C₄₂H₇₂O₁₆ | Flower buds of P. ginseng |
| 44 | Floralginsenoside D | C₄₁H₇₀O₁₅ | Flower buds of P. ginseng |
| 45 | Floralginsenoside E | C₄₂H₇₂O₁₅ | Flower buds of P. ginseng |
| 46 | Floralginsenoside F | C₄₂H₇₂O₁₅ | Flower buds of P. ginseng |
| 47 | Floralginsenoside G | C₅₀H₈₄O₂₁ | Flower buds of P. ginseng |
| 48 | Floralginsenoside I | C₄₈H₈₂O₂₀ | Flower buds of P. ginseng |
| 49 | Floralginsenoside K | C₄₈H₈₂O₂₁ | Flower buds of P. ginseng |
| 50 | Floralginsenoside O | C₅₃H₉₀O₂₄ | Flower buds of P. ginseng |
| 51 | Floralginsenoside P | C₅₃H₉₀O₂₃ | Flower buds of P. ginseng |
| 52 | Floralquinquenoside A | C₃₆H₆₂O₁₁ | Flower buds of P. quinquefolius |
| 53 | Floralquinquenoside C | C₄₂H₇₂O₁₅ | Flower buds of P. quinquefolius |
| 54 | Ginsenoside Rh6 | C₃₆H₆₂O₁₁ | Leaves of P. ginseng |
| 55 | Floralginsenoside La | C₄₈H₈₂O₁₉ | Flower buds of P. ginseng |
| 56 | Floralginsenoside Lb | C₄₈H₈₂O₁₉ | Flower buds of P. ginseng |
| 57 | Floranotoginsenoside D | C₅₃H₉₀O₂₃ | Flowers of P. notoginseng |
| 58 | Ginsenoside Rg7 | C₃₆H₆₀O₉ | Leaves of P. ginseng |
| 59 | Notopanaxoside A | C₃₆H₆₂O₁₀ | Roots of P. notoginseng |
| 60 | Notoginsenoside FT3 | C₄₇H₈₀O₁₈ | Acid hydrolysate roots of P. |
| | | | notoginseng |
| 61 | Floranotoginsenoside A | C₅₃H₉₀O₂₃ | Flowers of P. notoginseng |
| 62 | Ginsenoside ST2 | C₃₆H₆₂O₁₀ | Steamed leaves of P. ginseng |
| 63 | Notoginsenoside Rw2 | C₄₁H₇₀O₁₄ | Rhizomes of P. notoginseng |
| 64 | Notoginsenoside ST5 | C₄₇H₈₀O₁₈ | Steamed roots of P. notoginseng |
| 65 | Yesanchinoside H | C₅₃H₉₀O₂₃ | Underground part of P. japonicus |
| 66 | Ginsenoside Ki | C₃₆H₆₂O₁₀ | Leaves of P. ginseng |
| 67 | Ginsenoside Km | C₃₆H₆₂O₁₀ | Leaves of P. ginseng |
| 68 | Quinquenoside L2 | C₄₈H₈₂O₁₉ | Leaves and stems of P. quinquefolius |
| 69 | Dammar-25(26)-ene-3,6,12,20,22,24- | C₃₀H₅₂O₆ | Leaves of P. ginseng |
| | hexanol | | |
| 70 | Floralginsenoside Kb | C₄₅H₇₆O₁₉ | Flower buds of P. ginseng |
| 71 | Floralginsenoside Kc | C₄₅H₇₆O₂₀ | Flower buds of P. ginseng |
| 72 | Floralginsenoside Ta | C₃₆H₆₀O₁₀ | Flower buds of P. ginseng |
| 73 | Vina-ginsenoside R25 | C₄₂H₇₀O₁₅ | Roots and rhizomes of P. vietnamensis |
| 74 | Floralginsenoside Tb | C₃₅H₆₂O₁₁ | Flower buds of P. ginseng |
| 75 | Quinquenoside L9 | C₄₂H₇₄O₁₅ | Leaves and stems of P. quinquefolius |
| 76 | Quinquenoside L16 | C₅₄H₉₄O₂₅ | Leaves and stems of P. quinquefolius |
| 77 | 25-OCH₃-PPD | C₃₁H₅₆O₄ | Leaves of P. notoginseng |
| 78 | 25-OH-PPD | C₃₀H₅₄O₄ | Fruits of P. ginseng |
| 79 | 25-OH-PPT | C₃₀H₅₄O₅ | Fruits of P. ginseng |
| 80 | Notoginsenoside FT2 | C₄₇H₈₂O₁₈ | Acid hydrolysate roots of P. |
| | | | notoginseng |
| 81 | Notoginsenoside T4 | C₃₆H₆₂O₁₁ | Acid hydrolysate roots of P. |
| | | | notoginseng |
| 82 | Quinquenoside L1 | C₄₈H₈₀O₁₈ | Leaves and stems of P. quinquefolius |
| 83 | Quinquefoloside La | C₅₄H₉₂O₂₃ | Leaves of P. quinquefolius |
| 84 | Quinquefoloside Lc | C₅₄H₉₂O₂₃ | Leaves of P. quinquefolius |
| 85 | Dammar-(E)-20(22)-ene-3,12,25-triol | C₃₀H₅₂O₃ | Acid hydrolysate roots of P. ginseng |
| 86 | Notoginsenoside ST1 | C₃₆H₆₂O₁₀ | Steamed roots of P. notoginseng |
| 87 | Ginsenoside Rg6 | C₄₂H₇₀O₁₂ | Stem-leaves of P. ginseng |
| 88 | Ginsenoside Rs4 | C₄₂H₇₀O₁₂ | Steamed roots of P. notoginseng |
| 89 | Ginsenoside Rs6 | C₄₂H₇₀O₁₂ | Steamed roots of P. notoginseng |
| 90 | Isoginsenoside Rh3 | C₃₆H₆₀O₇ | Fruits of P. ginseng |
| 91 | Ginsenoside Rh5 | C₃₆H₆₀O₉ | Leaves of P. ginseng |
| 92 | Ginsenoside SL2 | C₄₂H₇₀O₁₄ | Steamed leaves of P. ginseng |
| 93 | Ginsenoside ST1 | C₃₆H₆₀O₁₀ | Steamed leaves of P. ginseng |
| 94 | Notoginsenoside ST2 | C₄₃H₇₄O₁₅ | Steamed roots of P. notoginseng |
| 95 | Notoginsenoside ST3 | C₄₃H₇₄O₁₅ | Steamed roots of P. notoginseng |
| 96 | Ginsenoside Rg8 | C₄₂H₇₀O₁₂ | Roots of P. quinquefolius |
| 97 | Notoginsenoside T1 | C₃₆H₆₀O₁₀ | Acid hydrolysate roots of P. |
| | | | notoginseng |
| 98 | Notoginsenoside T2 | C₃₆H₆₂O₁₀ | Acid hydrolysate roots of P. |
| | | | notoginseng |
| 99 | Ginsenoside Rg1-12,23-epoxy | C₄₂H₇₀O₁₄ | Leaves of P. ginseng |
| 100 | Ginsenoside Rh9 | C₃₆H₆₀O₉ | Leaves of P. ginseng |
| 101 | Quinquefoloside-Lb | C₅₃H₈₈O₂₂ | Leaves of P. quinquefolius |
| 102 | Ginsenoside Rk1 | C₄₂H₇₀O₁₂ | Processed roots of P. ginseng |
| 103 | Ginsenoside Rk2 | C₃₆H₆₀O₇ | Processed roots of P. ginseng |
| 104 | Ginsenoside Rk3 | C₃₆H₆₀O₈ | Processed roots of P. ginseng |
| 105 | Ginsenoside Rs5 | C₃₈H₆₂O₉ | Steamed roots of P. notoginseng |
| 106 | Ginsenoside Rs7 | C₃₈H₆₂O₉ | Steamed roots of P. notoginseng |
| 107 | Notoginsenoside T5 | C₄₁H₆₈O₁₂ | Acid hydrolysate roots of P. |
| | | | notoginseng |
| 108 | Ginsenoside Rz1 | C₄₂H₇₀O₁₂ | Steamed roots of P. notoginseng |
| 109 | Ginsenoside SL3 | C₄₂H₇₀O₁₄ | Steamed leaves of P. ginseng |
| 110 | Ginsenoside Rh8 | C₃₆H₆₀O₉ | Leaves of P. ginseng |
| 111 | Ginsenoside Rh7 | C₃₆H₆₀O₉ | Leaves of P. ginseng |
| 112 | Yesanchinoside G | C₅₃H₈₈O₂₃ | Underground part of P. japonicus |
| 113 | Yesanchinoside I | C₅₉H₁₀₀O₂₆ | Underground part of P. japonicus |
| 114 | Hexanordammaran | C₂₄H₄₀O₄ | Leaves of P. ginseng |
| 115 | Notoginsenoside R10 | C₃₀H₅₀O₉ | Steamed leaves of P. ginseng |
| 116 | Yesanchinoside A | C₄₄H₇₄O₁₆ | Underground part of P. japonicus |
| 117 | Yesanchinoside B | C₄₈H₈₂O₂₀ | Underground part of P. japonicus |
| 118 | Yesanchinoside C | C₄₇H₈₀O₁₉ | Underground part of P. japonicus |
| 119 | Panaxadione | C₃₀H₄₈O₅ | Seeds of P. ginseng |
| 120 | Polyacetyleneginsenoside Ro | C₆₅H₁₀₀O₂₁ | Roots of P. ginseng |
| 121 | Isodehydroprotopanaxatriol | C₃₀H₅₀O₃ | Acid hydrolysate roots of P. ginseng |
| 122 | 20,25-epoxy-dammaran-2-en-6,12-diol | C₃₀H₅₀O₃ | Acid hydrolysate roots of P. ginseng |
| 123 | 3-methyl-28-nordammaran-2-en-6,12-diol | C₃₀H₅₀O₃ | Acid hydrolysate roots of P. ginseng |

Analysis of ginseng root (Japanese ginseng) has indicated (per 100 grams root) 0.17 g (0.17%) total fat, 50 mg sodium, 8.82 g (8.82%) total carbohydrates comprising 2.3 g dietary fiber and 3.85 g sugars and 0.71 g (0.71%) protein content. Calorimetric calculation showed that 100 g of ginseng root contains 37 kcal.

According to a specific embodiment, the active ingredient or combination thereof includes a ginsenoside, e.g. a protopanaxadiol (PPD)-type saponin with sugar moieties attached to the C-3 and/or C-20, a protopanaxatriol (PPT) saponin with sugar moieties at C-6 and/or at C-20, an ocotillol-type saponin with a five-membered epoxy ring at C-20, an oleanane-type saponin with a nonsteroidal structure, and a dammarane type saponin.

Some specific ginsenosides include, but are not limited to notoginsenosides, yesanchinosides, panaxodione, floralginsenosides and ginsenosides Rg1, Rd, Re, Rb1, R1, Rg3, Rk1, Rf, Rg5, F4, Ro.

According to a specific embodiment, the active ingredient or combination thereof includes a volatile compound, e.g., terpene hydrocarbons, monoterpene and sesquiterpene hydrocarbons, specifically β-alamene and β-selenine.

According to a specific embodiment, the active ingredient or combination thereof includes a phytosterol, e.g., stigmasterol, beta-sterol.

According to a specific embodiment, the active ingredient or combination thereof includes a polyacetylene, e.g., panaxynol, ginsenoyne A.

According to a specific embodiment, the active ingredient or combination thereof includes a flavenoid, e.g., Kaempferol.

According to a specific embodiment, the active ingredient or combination thereof includes an alkaloid, e.g., fumarine, girinimbin.

According to a specific embodiment, the active ingredient or combination thereof includes a polysaccharide, e.g., WGP, KGP-1, KGP-4, WGPE, NGP, EGP.

According to a specific embodiment, the active ingredient or combination thereof includes a phenolic compound, e.g., elemicin, dauricin, maltol.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., vitamin D, vitamin A and vitamin C.

According to a specific embodiment, a methanol or ethanol extract is performed, e.g., ethanol concentration is 80%; extraction time is 24 h; extraction temperature is 80-90° C.; particle size 1.0 mm; and solvent to ginseng ratio of 20:1 ml/g. Other extraction procedures include, but are not limited to, those described in Dong et al. 2017 Phytother Res Aug; 19(8): 684-688, which is hereby incorporated by reference in its entirety.

According to another embodiment, the plant part is leaf.

Also contemplated herein are plants of the genus Panax.

Examples include hut are not limited to

| | Common name and |
|---|---|
| | geographical |
| Ginseng Species | designation |
| P. gensing | Korean ginseng |
| P. guinquefolius | American ginseng |
| P. notoginseng | Chinese ginseng |
| P. japonicas | Japanese ginseng |
| P. omiensis | Omei gensing |
| P. pseudoginseng | Himalayan ginseng |
| P. assamicus | N/A |
| P. shangianus | N/A |
| P. sinensis | N/A |
| P. stipuleanatus | Pingpien ginseng |
| P. trifolius | Dwarf ginseng |
| P. variabilis | N/A |
| P. vietnamensis | Vietnamese ginseng |
| P. wangianus | Narrow-leaved |
| P. bipinnatifidus | Feather-leaf bamboo |
| | ginseng |
| P. sokpayensis | N/A |
| P. zingiberensis | Ginger ginseng |

Korean ginseng cultivars suitable for use with the present invention include, but are not limited to: Chunpoong, Yunpoong, Gopoong, Sunpoong, Gumpoong, Cheongsun, Sunhyang, Sunun, Sunone, K-1, G-1 and Kowon. Chinese ginseng cultivars suitable for use with the present invention include, but are not limited to Jilin Huangguo Reshen, Jishen 01, Fuxing 01, Fuxing 02, Kangmei 01, Xinkaihe 01, Xinkaihe 02, Zhongnong Huangfengshen and Zhongda Linxiashen.

Chemical Composition of Boswellia Species (Frankincense, Olibanum)

Olibanum, also known as frankincense, is a natural oleo-gum-resin that exudes from tappings in the bark of Boswellia trees. There are approximately 23 species of trees in the genus Boswellia, which grow mainly in Arabia, on the eastern coast of Africa and in India. Characterization and identification of chemical compounds of Olibanum using a variety of methods identified a large variety of compounds in the gum resin of Boswellia tree species and classified them as generally being:
Alcohol-soluble resins (e.g. diterpenes, triterpenes)
Highly aromatic essential oils (e.g. mono- and sesquiterpenes)
Water soluble gums

According to specific embodiments, Olibanum comprises 65-85% alcohol-soluble resins, about 5-9% highly aromatic essential oils and the remainder water soluble gums.

In India, the main commercial sources of Boswellia serrata are Andhra Pradesh, Gujarat, Madhya Pradesh, Jharkhand and Chhattisgarh. Regionally, it is also known by different names. The botanical origin and vernacular names of Boswellia serrata are given in below Table 1. Salai, an oleo gum-resin, is a plant exudate of genus Boswellia (Family: Burseraceae). It is tapped from the incision made on the trunk of the tree, which is then stored in specially made bamboo basket. The semi-solid gum-resin is allowed to remain in the basket for about a month during which its fluid content locally known as ‘ras’ keeps flowing out. The residue, semi-solid to solid part, is the gum-resin which hardens slowly into amorphous, tear-shaped products with an aromatic scent. Then, it is broken into small pieces by wooden mallet or chopper and during this process all impurities including bark pieces etc. are removed manually. The gum-resin is then graded according to its flavour, colour, shape and size. Generally four grades i.e. Superfine, Grade I, Grade II and Grade III are available in the market. The fresh gum obtained from the tree is hot with pleasant flavour and slightly bitter in taste. It had been the ‘frankincense’ of ancient Egyptians, Greeks and Romans who used it as prized incense, fumigant as well as a multipurpose aromatic. It is generally used in making incense powder and sticks.

TABLE 1
BOTANICAL ORIGIN AND VERNACULAR
NAMES OF BOSWELLIA SERRATA

| Botanical origin | Vernacular names |
|---|---|
| Division: Spermatophyta | English: Indian Olibanum or Indian |
| | frankincense |
| Sub-division: Anglospermae | Hindi: Kundur, Salai |
| Tribe: Rosopsida | Bengali: Kundur, Salai |
| Sub-tribe: Rosidae S. lat. | Gujarati: Dhup, Gugali |
| Over-class: Rutanae | Kannada: Chitta, Guguladhuph |
| Class: Anacardiales | Malayalam: Parangi, Saambraani |
| Family: Burseraceae | Tamil: Parangi, Saambraani |
| Genus: Boswellia | Telugu: Phirangi, Saambraani |
| Species: serrata | Sanskrit: Ashvamutri, Kundara, Shallaki. |

The oleo gum-resins contain 30-60% resin, 5-10% essential oils, which are soluble in the organic solvents, and the rest is made up of polysaccharides (˜65% arabinose, galactose, xylose) which are soluble in water. The resins have a fragrant aroma because of the presence of essential oils and this accounts for their commercial importance.

According to specific embodiments, the common components of Olibanum belonging to the terpene and sesquiterpene families, or their terpenoid derivatives include, but are not limited to α- and β-pinene, α-limonene, myrcene, linalool, α-cubebene, γ-cadinene, β-bourbonene, and α-phellandrene dimer compounds in Olibanum are the compounds that constitute its phytochemical activity. Several oxygenated isoprenoid derivatives have also been identifed, such as carbonyl derivatives (e.g., carvone, fenchone) and alcohol-containing terpene and sesquiterpene derivatives (e.g., transpinocarveol, cis-verbenol, and cembrenol), as well as ester-containing compounds (e.g., α-terpinyl acetate and bornyl acetate).

Diverse investigators have reported that limonene is the most abundant volatile in Olibanum, while others have identified octanol acetate, α-pinene and α-thujene as most abundant depending on the species of Boswellia plant material used for extraction.

More than 300 essential oils have been isolated from Boswellia ssp.

The table below shows the essential oils recovered from Olibanum extracts prepared by different extraction procedures, from diverse Boswellia ssp.:

| Number | Compound |
|---|---|
| 1 | 5,5-Dimethyl-1-vinylbicyclo-[2.1.1]-hexane |
| 2 | Anethol |
| 3 | Benzyl tiglate |
| 4 | trans-α-Bergamotene |
| 5 | Bornyl acetate |
| 6 | β-Bourbonene |
| 7 | Cadinene |
| 8 | γ-Cadinene |
| 9 | Camphene |
| 10 | Camphor |
| 11 | m-Camphorene |
| 12 | p-Camphorene |
| 13 | Carene-3 |
| 14 | (E)-β-Caryophyllene |
| 15 | Cembrene A |
| 16 | Cembrenol |
| 17 | 1,8 Cineol |
| 18 | Citronellol |
| 19 | α-Copaene |
| 20 | β-Copaene |
| 21 | p-Cymene |
| 22 | m-Cymene |
| 23 | Elemol |
| 24 | Elemicine |
| 25 | epi-Cubenol |
| 26 | Estragol |
| 27 | Eudesmol |
| 28 | 10-epi-γ-Eudesmol |
| 29 | Fenchone |
| 30 | Geraniol |
| 31 | Germacrene D |
| 32 | Humulene epoxide |
| 33 | Isoincensole |
| 34 | Isomenthone |
| 35 | Kessane |
| 36 | Limonene |
| 37 | Linalool |
| 38 | Linalyl acetate |
| 39 | Menthone |
| 40 | Methylchavicol |
| 41 | Methylisoeugenol |
| 42 | Methyleugenol |
| 43 | γ-Muurolene |
| 44 | Myrcene |
| 45 | Neocembrene A |
| 46 | Nerolidol |
| 47 | cis-β-ocimene |
| 48 | (Z)-Ocimene |
| 49 | (E)-β-Ocimene |
| 50 | Perillene |
| 51 | α-Phellandrene |
| 52 | β-Phellandrene |
| 53 | α-Pinene |
| 54 | β-Pinene |
| 55 | trans-Pinocarveol |
| 56 | Sabinene |
| 57 | cis-Sabinol |
| 58 | Terpinin-4-ol |
| 59 | Terpinen-4-ol |
| 60 | Terpinolene |
| 61 | α-Terpineol |
| 62 | α-Terpinene |
| 63 | α-Terpinene |
| 64 | γ-Terpinene |
| 65 | Terpinyl acetate |
| 66 | Terpinyl isobutyrate |
| 67 | Tetrahydrolinalool |
| 68 | α-Thujene |
| 69 | α-Thujone |
| 70 | β-Thujone |
| 71 | Tricyclene |
| 72 | Undecenol |
| 73 | trans-Verbenol |
| 74 | β-Ylangene |
| 75 | Zingiberene |
| 76 | Abieta-8,12-diene |
| 77 | α-Amorphene |
| 78 | alloaromadendrene |
| 79 | Benzyl benzoate |
| 80 | Beyerene |
| 81 | Bisabolene |
| 82 | Isopentyl-2-methylbutanoate |
| 83 | cis-Calamenene |
| 84 | α-Cadinene |
| 85 | τ-Cadinol |
| 86 | 2-Carene |
| 87 | Campholenealdehyde |
| 88 | Caryophyllene oxide |
| 89 | cis-Carveol |
| 90 | (+) trans-Carveol |
| 91 | Carvone |
| 92 | α-Cedrene |
| 93 | Cedrol |
| 94 | Cembra-1,3,7,11-tetraene |
| 95 | Cembra-3,7,11,15-tetraene |
| 96 | Cembrene |
| 97 | Cembrene C |
| 98 | Citronellyl acetate |
| 99 | α-Cubebene |
| 100 | β-Cubebene |
| 101 | o-Cymene |
| 102 | Chrysanthenone |
| 103 | 1,4-Cyclohexadiene |
| 104 | p-Cymen-8-ol |
| 105 | Decanol |
| 106 | Decyl acetate |
| 107 | 2,6-Dimethoxytoluene |
| 108 | 3,5-Dimethoxytoluene |
| 109 | Duva-3,9,13-trien-1,5α-diol |
| 110 | Duva-4,8,13-trien-1a,3α-diol |
| 111 | Duva-3,9,13-trien-1,5α-diol-1-acetate |
| 112 | Duva-3,9,13-triene-1α-ol-5,8-oxide-1-acetate |
| 113 | β-Elemene |
| 114 | Farnesyl acetate |
| 115 | Geranyl acetate |
| 116 | α-Gurjunene |
| 117 | Hedycariol |
| 118 | 1,3,6-Trimethylencycloheptane |
| 119 | 1-Hexanol |
| 120 | Hexyl acetate |
| 121 | Hexyl hexanoate |
| 122 | α-Humulene |
| 123 | Incensole |
| 124 | Incensole acetate |
| 125 | Isodurene |
| 126 | Isocembrene |
| 127 | Isophyllocladene (kaur-15-ene) |
| 128 | Kaurene |
| 129 | Ledol |
| 130 | Maaliane |
| 131 | p-Mentha-1,5-dien-8-ol |
| 132 | o-Methyl anisole |
| 133 | α-Muurolene |
| 134 | α-Muurolol |
| 135 | Myrtenal |
| 136 | Naphthalene |
| 137 | Naphthalene 1,2,3,4,4a,7-hexahydro-1,6-dimethyl-4- |
| | (1-methylethyl |
| 138 | Neryl acetate |
| 139 | cis-Nerolidol |
| 140 | (S)-trans-Nerolidol |
| 141 | (E)-Nerolidol |
| 142 | 1-Octanol |
| 143 | n-Octanol |
| 144 | Octanol acetate |
| 145 | Octyl acetate |
| 146 | Octyl formate |
| 147 | allo-Ocimene |
| 148 | Phenanthrene-7-ethenyl-9,10,10a-dodeca-hydro-1-1-4a- |
| | 7-tetramethyl |
| 149 | α-Phellandrene epoxide |
| 150 | Phyllocladene |
| 151 | α-Pinene-epoxide |
| 152 | 1-β-Pinene |
| 153 | 2-β-Pinene |
| 154 | Isopinocampheol |
| 155 | Piperitone |
| 156 | Pyrimidine |
| 157 | Sabinyl acetate |
| 158 | Sandaracopimara-8(14)-15-diene |
| 159 | Sclarene |
| 160 | α-Selinene |
| 161 | β-Selinene |
| 162 | δ-Selinene |
| 163 | trans-Terpine |
| 164 | 4-Terpineol |
| 165 | Terpinolene |
| 166 | Isoterpinolene |
| 167 | 2,4(10)-Thujadiene |
| 168 | Thujopsene |
| 169 | Thunbergol |
| 170 | Isomyl-valerate |
| 171 | Verticilla-4(20),7,11-triene |
| 172 | Verbenone |
| 173 | cis-Verbenol |
| 174 | Verticiol |
| 175 | Viridiflorol |
| 176 | Benzene, 1methoxy-2-methyl |
| 177 | endo-Borneol |
| 178 | γ-Campholene aldehyde |
| 179 | α-Campholene aldehyde |
| 180 | Cara-2,4-diene |
| 181 | Carvacrol |
| 182 | Carvotanacetone |
| 183 | trans-Dihydrocarvone |
| 184 | Cumin alcohol |
| 185 | m-Cymene-8-ol |
| 186 | p-Cymene-9-ol |
| 187 | p-Cymenene |
| 188 | Dodecanol |
| 189 | Eucalyptol |
| 190 | Eucarvone |
| 191 | Isopropyl benzaldehyde |
| 192 | Isopropyl benzalcohol |
| 193 | cis-1,2-Limonene epoxide |
| 194 | 8,9-Limonene epoxide II |
| 195 | 8,9-Limonene-epoxide I |
| 196 | trans-1,2-Limonene epoxide |
| 197 | cis-Linalool oxide |
| 198 | trans-Linalool oxide |
| 199 | p-Mentha-1,5-diene-7-ol |
| 200 | p-Mentha-1,8-diene-4-ol |
| 201 | cis-p-Menth-2-en-1-ol |
| 202 | cis-p-Mentha-1(7),8-diene-2-ol |
| 203 | cis-p-Mentha-2,8-diene-1-ol |
| 204 | trans-p-Menth-2-en-1-ol |
| 205 | trans-p-Mentha-1(7),8-diene-2-ol |
| 206 | trans-p-Mentha-2,8-diene-1-ol |
| 207 | 2,4(8)-p-Menthadiene |
| 208 | p-Mentha-6,8-dien-2-one |
| 209 | p-Methylanisole |
| 210 | Myrtenol |
| 211 | Nerol |
| 212 | trans-Ocimene |
| 213 | (E)-β-Ocimene epoxide |
| 214 | α-Phellandrene-dimer |
| 215 | α-Phellandrene-8-ol |
| 216 | α-Pinene oxide |
| 217 | Pinocamphone |
| 218 | Pinocarvone |
| 219 | Piperitenone |
| 220 | Isopiperitenone |
| 221 | trans-Piperitol |
| 222 | α-Terpineol |
| 223 | Sabina ketone |
| 224 | cis-Sabinene hydrate |
| 225 | trans-Sabinene hydrate |
| 226 | trans-Sabinol |
| 227 | 2,5-Dimethylstyrene |
| 228 | cis-1,2-Epoxy-terpin-4-ol |
| 229 | Thuj-3-en-10-al |
| 230 | Thujanol |
| 231 | Thunbergene |
| 232 | Thymol |
| 233 | Umbellulone |
| 234 | Verticellol |
| 235 | 5,5-Dimethyl-1-vinylbicyclo-[2.1.1]-hexane |
| 236 | p-Anisaldehyde |
| 237 | Aromadendrene |
| 238 | Benzyl tigilate |
| 239 | p-Camphorene |
| 240 | Isocaryophyllene |
| 241 | Cumaldehyde |
| 242 | Cyclosativene |
| 243 | γ-Eudesmol |
| 244 | Guaioxide |
| 245 | 5-Guaiene-11-ol |
| 246 | Isogermacrene D |
| 247 | 4-Methylene-1-(1-methylethyl)-bicyclo[3.1.0]hex-2-ene |
| 248 | 2-Methyl-5-(1-methylethyl)-1,3-cyclohexadiene |
| | monoepoxide |
| 249 | n-Pentadecan |
| 250 | Perilla alcohol |
| 251 | Perillol |
| 252 | Thujol |
| 253 | m-Thymol |
| 254 | α-Ylangene |
| 255 | γ-Campholene aldehyde |
| 256 | n-Decanoic acid |
| 257 | β-Eudesmene |
| 258 | β-Cyclogeranylacetate |
| 259 | n-Hexanoic acid |
| 260 | Hexylcaprylate |
| 261 | Incensyl acetate |
| 262 | Incensole oxide |
| 263 | Incensole oxide acetate |
| 264 | Lauric acid |
| 265 | p-Methylacetophenone |
| 266 | p-Methyleugenol |
| 267 | β-Myrcene |
| 268 | n-Nonanoic acid |
| 269 | n-Octanoic acid |
| 270 | 3,4-Dimethoxystyrene |
| 271 | α-Cadinol |
| 272 | 1,Hydroxy-1,7-dimethyl-4-isopropyl-2,7-cyclodecadiene |
| 273 | 1,5,5,8-Tetramethyl-12-oxabicyclo-[9.1.0]-dodeca-3,7-diene |
| 274 | 1-Methyl-4-(1-methylethenyl)-1,2-cyclohexanediol |
| 275 | trans-p-Mentha-2,8-dienol |
| 276 | 1,2,3,4,6,8a-hexahydro-1-isopropyl-4,7-dimethyl- |
| | naphthalene |
| 277 | 2-Isopropenyl-4a,8-dimethyl-1,2,3,4,4a,5,6,8a- |
| | ctahydronaphthalene |
| 278 | 3,5-Dimethoxytoluene |
| 279 | (Z)-α-Hydroxymanool |
| 280 | Hydroxy-manool |
| 281 | Methyl linoleate |
| 282 | 1-Acetyl-4-isopropenylcyclopentene |
| 283 | 2,4-Dimethylacetophenone |
| 284 | α-Amyrenone |
| 285 | β-Amyrenone |
| 286 | 10-Hydroxy-4-cadinen-3-one |
| 287 | 2-Hydroxy-1,4-cineole |
| 288 | Cryptone |
| 289 | Eucarvone |
| 290 | Isopropylidencyclohexane |
| 291 | 1,2,4-Trihydroxy-p-menthane |
| 292 | Δ⁴-p-Menthen-2-one |
| 293 | 5-Hydroxy-p-menth-6-en-2-one |
| 294 | Myrtenoic acid |
| 295 | Nopinone |
| 296 | 3,6,6,-Trimethyl-norpinan-2-one |
| 297 | o-Methylacetophenone |
| 298 | Perillaaldehyde |
| 299 | Phellandra |
| 300 | Pinocamphone/isopinocamphone |
| 301 | Thujone |
| 302 | 24-Noroleana-3,12-diene |
| 303 | 24-Noroleana-3,9(11),12-triene |
| 304 | 24-Norursa-3,12-diene |
| 305 | 24-Norursa-3,9(11),12-triene |
| 306 | 24-Norursa-3.12-dien-11-one |
| 307 | α-Amyrine |
| 308 | epi-α-Amyrine |
| 309 | β-Amyrine |
| 310 | Lupeol |
| 311 | Terpinenyl acetate |
| 312 | 1,5-Isopropyl-2-methylbicyclo[3.1.0]hex-3-en-2-ol |
| 313 | α-Campholenal |
| 314 | (3E,5E)-2,6-Dimethyl-1,3,5,7-octatetraene |
| 315 | (E)-2,3-Epoxycarene |
| 316 | 3,4-Dimethylstyrene |
| 317 | 1-(2,4-Dimethylphenyl)ethanol |
| 318 | 4-Methylbenzoic acid |
| 319 | p-Menth-1(7)-en-2-one |
| 320 | Caryophyllene |
| 321 | Methylcycloundecanecarboxylate |
| 322 | Nonanoic acid |
| 323 | Hexadecanoic acid |
| 324 | 1,4-Cineol |
| 325 | Sabinene hydrate |
| 326 | Methyl-trans-2-cis-4-decadienoate |
| 327 | 2-Hydroxy-5-methoxy-acetophenone |
| 328 | (E)-β-Farnesene |
| 329 | 2-Dodecenoic acid methyl ester |
| 330 | Calacorene |
| 331 | n-Dodecanoic acid |
| 332 | α-Guaiol |
| 333 | Caryophylla-3(15),7(14)-dien-6-ol |
| 334 | Cadalene |
| 335 | Eudesma-4(15),7-dien-1β-ol |
| 336 | n-Heptadecane |
| 337 | n-Tetradecanoic acid |
| 338 | n-Octadecane |
| 339 | Galaxolide |
| 340 | Manool |

Although many Boswellia species produce Olibanum, the major sources of commercial Olibanum are B. serrata (India), B. sacra (Oman), and B carteri (Somalia). The table below shows the major components of Olibanum derived from diverse Boswellia species, according to their percentage representation:

| | | Predominant | Percentage |
|---|---|---|---|
| Boswellia specie | Source of resin | compound(s) | (%) |
| B. serrata | Commercial (Hamburg, Germany) | Myrcene | 38 |
| B. serrata | NA | α-Thujene | 22.7-47.4 |
| B. serrata | NA | α-Thujene | 29.3 |
| B. serrata | NA | α-Thujene | 61.36 |
| B. carteri | Purchased from the local market of | Duva-3,9,13-triene- | 21.4 |
| | herbs and spices in Egypt | 1a-ol-5,8-oxide-1- | |
| | | acetate | |
| B. sacra | Botanically certified oleogum resin | E-β-Ocimene | 32.3 |
| B. carteri/sacra | NM | Octanol acetate | 45.2 |
| B. carteri | Authentic sample from Ethiopia | Octyl acetate | 39.3 |
| | certified for its authenticity from the | | |
| | Agricultural Department of the | | |
| | Ethiopian government | | |
| B. rivae | NA | Limonene | 28.0 |
| B. rivae | Authentic sample from Ethiopia | α-Pinene | 16.7 |
| B. rivae | NA | α-Pinene | 13.3 |
| B. rivae | NA | Octanol | 17.8 |
| B. neglecta | NA | α-Pinene | 16.7 |
| B. neglecta | Authentic sample from Ethiopia | α-Pinene | 21.3 |
| B. papyrifera | NA | Octyl acetate | 63.5 |
| B. papyrifera | NA | Octyl acetate | 56.0 |
| B. pirottae | NA | Trans-Verbenol | 15.5 |
| B. pirottae | NA | Terpinen-4-ol | 14.6 |
| B. frereana | Commercial (Hamburg, Germany) | α-Pinene | 38.0 |

One exemplary analysis of Olibanum has indicated the following components
Acid resin (6%), soluble in alcohol and having the formula C₂₀H₃₂O₄
gum (similar to gum arabic) 30-36%
3-acetyl-beta-boswellic acid (Boswellia sacra)
alpha-boswellic acid (Boswellia sacra)
incensole acetate, C₂₁H₃₄O₃
phellandrene

Another analysis of B. serrata resin revealed that the resinous part of Boswellia serrata contains monoterpenes (α-thujene); diterpenes (macrocyclic diterpenoids such as incensole, incensole oxide, iso-incensole oxide, a diterpene alcohol [serratol]); triterpenes (such as α- and β-amyrins); pentacyclic triterpenic acids (boswellic acids); tetracyclic triterpenic acids (tirucall-8,24-dien-21-oic acids). The structures of four major pentacyclic triterpenic acids (boswellic acids) as also some of their characteristic features of four pentacyclic triterpene acids (Boswellic acid) are given in the following table:

| | | | 11-keto-β-Boswellic | Acetyl-11-keto-β-Boswellic |
|---|---|---|---|---|
| Properties | β-Boswellic acid | Acetyl-β-Boswellic acid | acid | acid |
| Molecular formula | C₃₀H₄₈O₃ | C₃₂H₅₀O₄ | C₃₀H₄₆O₄ | C₃₂H₄₈O₅ |
| Molecular weight | 456.7 | 498.74 | 470.69 | 512.73 |
| Chemical name | 3α-Hydroxy-urs-12-en- | 3α-Acetoxy-urs-12-en-23- | 3α-Hydroxy-urs-12-en-11- | 3α-Acetoxy-urs-12-en-11- |
| | 23-oic acid | oic acid | keto-23-oic acid | keto-23-oic acid |
| Melting point | 226-228° | 252-255° | 195-197° | 271-274° |
| Specific rotation | +106.8° | +138° | +78.5° | +88.5° |
| UV-MeOH | Maxima at 208 nm | Maxima at 208 nm | Maxima at 250 nm | Maxima at 250 nm |
| NMR (in CDCl₃, δ ppm) | 5.15, CH═C; 4.08, CH—OH; | 5.31, CH═C; 5.2, CH—OAc; | 5.55, CH═C; 4.08, CH—OH; | 5.55, CH═C; 5.2, CH—OAc; |
| | 2.3-1.1, Methylenes and | 2.1, COCH_{3;} 1.9-1.25, | 2.6-1.4, Methylenes and | 2.6-1.4, Methylenes and |
| | methines, 23 protons; 1.1- | Methylenes and methines | methines 21 protons; | methines 21 protons; 1.25- |
| | 0.7 Methyls, 21 protons | 23 protons; 1.2-0.7, | 1.25-0.75, Methyls 21 | 0.75, Methyls 21 protons |
| | | Methyls 21 protons | protons | |
| FTIR (in KBr, cm⁻¹) | 3500 (OH), 1699.5 (COOH) | 1732 (OAc), 1701 (COOH) | 3460 (OH), 1693 (COOH) | 1740 (Ac), 1701 (COOH), |
| | | | | 647(α,β-unsaturated |
| | | | | carbonyl). |
| GC-MS | 394 (M-68[44 due to —CO₂ | 394 (M-104[44 due to —CO₂ | 408 (M-68[44 due to—CO₂ | 408 (M-68[44 due to—CO₂ |
| | and 18 due to —H₂O]); Other | and 60 due to —HOAc]); 218 | and 18 due to—H₂O]); 232 | and 18 due to —HOAc]); |
| | fragments: 203, 189, 175, | (base peak) | (base peak); | 232 (base peak); Other |
| | 161. | | Other fragments: 217, 175 | fragments: 217, 175, |
| | | | 161, 135 | 161, 135 |

The Olibanum gum component contains polysaccharides and polymeric components. The proteoglycans in Olibanum comprise mainly D-galactose units in the main chain and glucuronic acid, uronic acids, 4-O-methyl-glucuronic acid and arabinose in the side chains.

According to a specific embodiment, the active ingredient or combination thereof includes an alcohol soluble acid resin, a water soluble gum, an alpha-boswellic acid, an incensole acetate and a phellandrene.

According to a specific embodiment, the active ingredient or combination thereof includes a volatile compound, e.g. α-Thujene, Duva-3,9,13-triene-1a-ol-5,8-oxide-1-acetate, E-β-Ocimene, Octanol acetate, Octyl acetate, Limonene, α-Pinene, Octanol, Trans-Verbenol and Terpinen-4-ol.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, a water or alcohol extract is performed.

In some embodiments, the Olibanum is prepared by water extract. An exemplary water extract is described herein:

Preparation of olibanum extract by water. At first, Olibanum is carefully powdered. The powder (25 g) is mixed with 200 ml of deionized water and stirred with 800 rpm overnight at room temperature. This mixture is centrifuged at 1,500 rpm for 10 min and the supernatant collected. Thereafter, the supernatant is again centrifuged at 2,500 rpm for 10 min and successively at 10,000 rpm for 20 min, and then filtered. The filtrates can be stored at −20 C and then freeze-dried −58 C and 0.5 Torr for 24 h to yield 4.02 gr of water soluble extract. At the next step, the resulted powder is dissolved in 100 ml methanol and stirred for 12 hr. at room temperature, then allowed to settle. The precipitate phase is collected and dried in an oven. Again the powder is dissolved in deionized water, centrifuged repeatedly and refiltered. The filtrates can be stored and then freeze-dried.

In some embodiments, the Olibanum is prepared by alcohol extract. An exemplary alcohol extract is described herein:

Preparation of olibanum extract by alcohol: In this method, 100 gr of Olibanum powder with 400 ml of methanol is mixed. This mixture is then stirred at 650 rpm for 24 hours. The resulting mixture is made up of two phases, the upper phase is alcoholic and yellow, and contains substances that are soluble in alcohol. The material is then dried in an oven at 50 C. The bottom phase has a sedimentary and white state, which is set to in the oven until dry. The resulting powder in the water is well dissolved and the obtained solution is centrifuged at 1,500 rpm for 10 min and the supernatant collected. Thereafter, the supernatant is again centrifuged at 2,500 rpm for 10 min and successively at 10,000 rpm for 20 min, and then filtered. The filtrates can be stored at −20 C and then freeze-dried.

Other extraction procedures include, but are not limited to, those described in Mertens et al, et al. 2009, Flavor and Fragrance, 24:279-300 and Hamm et al, Phytochemistry 2005, 66:1499-1514, which are hereby incorporated by reference in their entirety.

Also contemplated herein are Olibarum and other compositions from trees of the genus Boswellia.

Examples include, but are not limited to:

Some Boswellia
Species
B. socotrana
B. elongata
B. ameero
B. carteri
B. neglecta
B. sacra
B. thurifera
B. frereana
B. dioscorides
B. rivae
B. papyrifera
B. serrata

Chemical Composition of Gynostemma pentaphyllum (Jiaogulan)

Gynostemma pentaphyllum is a perennial herb from the Cucurbitaceae family, with 5-lobed leaves and a gourd-like, inedible fruit which grows in forests, thickets or roadsise on mountain slopes in many areas of Northeast and Southeast Asia, including China,

Taiwan, S Korea, Japan, Thailand, Vietnam and Laos. G. pentphyllum also grows in Bangladesh, Bhutan, India, Indonesia, Malaysia, Myanmar, Nepal, New Guinea and Sri Lanka. Jiaogulan is prized for its reputation as a “longevity plant”. Characterization and identification of chemical compounds of Gynostemma pentaphyllum using a variety of methods identified a large variety of compounds in Gynostemma pentaphyllum (Thun.) Makino and classified them as generally being:
Saponin Glycosides (e.g., gypenosides)
Phenolic compounds
Flavenoids (e.g. Kaempferol, quercetin, rutin, ombuin, isorahmnetin)
Polysaccharides
Sterols (e.g. ergostane, cholestane, stigmastane)
Trace elements (e.g. Cu, Fe, Zn, Mn, Co, Ni, Se, Mo and Sr)
Carotenoids
Volatiles (e.g. malonic acid, benzyl-O-beta-D-glucopyranoside, lutein, vomifoliol, palmitic acid, linoleic acid)

According to specific embodiments, the saponin compounds in Jiaogulan and the polysaccharide compounds are the compounds that constitute its phytochemical activity. The most abundant saponin compound in Jiaogulan was found to be gypenoside.

Most Jiaogulan saponins belong to a family of triterpenoid saponins. They are also referred to as gypenosides, and dammarane derivatives. More than 150 saponins have been isolated from G. pentaphyllum plants. Saponins have been identified in Jiaogulan leaves and stems, flower buds, fruits, berries, and seeds.

The table below shows the phytochemical properties of 5 different Gynostemma pentaphyllum samples from different sources:

| | | TPC | TSC | TFC | RUTIN | QUERCITIN | R + Q |
|---|---|---|---|---|---|---|---|
| | | (mg | (mg | (mg | CONTENT | CONTENT | (umol |
| SAMPLE | SOLVENT | GAE/g) | GE/g) | RE/g) | (ug/g) | (ug/g) | QE/g) |
| GP1 | 50% acetone | 44.3 | 38.02 | 21.44 | 3049.5 | 4906.5 | 21.2 |
| | 50% ethanol | 37.5 | 41.39 | 26.40 | 7948.2 | 7431.8 | 37.6 |
| | 100% | 33.6 | 87.28 | 26.87 | 11235.4 | 7279.1 | 42.5 |
| | ethanol | | | | | | |
| GP2 | 50% acetone | 14.9 | 90.17 | 10.6 | 2527.3 | 117.5 | 4.5 |
| | 50% ethanol | 12.9 | 114.48 | 14.27 | 3588.1 | 136.2 | 6.3 |
| | 100% | 6.9 | 132.57 | 13.84 | 2131.9 | 166.2 | 4.0 |
| | ethanol | | | | | | |
| GP3 | 50% acetone | 12.3 | 47.62 | 10.52 | 8614.9 | 358.9 | 15.3 |
| | 50% ethanol | 10.6 | 59.13 | 9.51 | 9954.0 | 411.0 | 17.7 |
| | 100% | 6.7 | 64.57 | 8.05 | 7193.0 | 549.4 | 13.6 |
| | ethanol | | | | | | |
| GP4 | 50% acetone | 43.2 | 77.64 | 63.48 | 1409.2 | 241.3 | 3.1 |
| | 50% ethanol | 30.4 | 82.12 | 54.04 | 680.2 | 150.8 | 1.6 |
| | 100% | 17.7 | 104.1 | 36.47 | 579.4 | 151.3 | 1.4 |
| | ethanol | | | | | | |
| GP5 | 50% acetone | 13.1 | 23.61 | 14.55 | nd | nd | |
| | 50% ethanol | 10.2 | 60.7 | 16.53 | nd | nd | |
| | 100% | 8.9 | 123.97 | 22.11 | nd | nd | |
| | ethanol | | | | | | |

GP1-5 represent G. pentaphyllum samples from different sources. Data are per gram of dry botanical basis and are expressed as mean (SD. Different letters represent significant differences (P < 0.05). nd stands for not detectable. TPC, TSC, and TFC stand for total phenolic content, total saponin content, and total flavonoid content by spectrometric methods, respectively. GAE, GE, RE, and QE stand for gallic acid equivalents, gypenoside equivalents, rutin equivalents, and quercetin equivalents. Rutin and quercetin contents were flavonoid profile obtained by HPLC. R + Q stands for total amount of rutin and quercetin.
Ethanol extraction: 12 g sample in 250 ml 100% ethanol, 5 hours in Soxhlet apparatus. 50% acetone extraction and 75% ethanol extraction: 2 g sample in 20 ml solvent at ambient temperature and filtration through 45 micron filter.

Water content of the Jiaogulan samples ranged from 3.79 to 7.57 g/100 g sample. Dietary fiber content ranged from 0.6 g/g to 0.24 g/g sample. Selenium content ranged from 1.7 mg/kg to 0.94 mg/kg.

According to a specific embodiment, the active ingredient or combination thereof includes a gypenoside. Some specific gypenosides include, but are not limited to CP-1-6.

According to a specific embodiment, the active ingredient or combination thereof includes a volatile compound, e.g., malonic acid, benzyl-O-beta-D-glucopyranoside, lutein, vomifoliol, palmitic acid, linoleic acid.

According to a specific embodiment, the active ingredient or combination thereof includes a phytosterol, e.g., stigmasterol, ergostane.

According to a specific embodiment, the active ingredient or combination thereof includes a flavenoid, e.g., Kaempferol, quercetin, rutin.

According to a specific embodiment, the active ingredient or combination thereof includes a phenolic compound.

According to a specific embodiment, the active ingredient or combination thereof includes a mineral, e.g., potassium, calcium, magnesium, phosphorus, aluminum, iron, sodium, boron, zinc, cadmium, selenium.

According to a specific embodiment, the active ingredient or combination thereof includes a vitamin, e.g., vitamin D, vitamin A and vitamin C.

According to a specific embodiment, a methanol or ethanol extract is performed, e.g., ethanol concentration is 100 or 75%; 5 hours in Soxhlet apparatus, or 50% acetone extraction and 75% ethanol extraction: 2 g sample in 20 ml solvent at ambient temperature and filtration through 45 micron filter. Other extraction procedures include, but are not limited to, those described in Yantao et al. 2016 Chi Med 11:43, which is hereby incorporated by reference in its entirety.

According to another embodiment, the plant part is leaf.

Also contemplated herein are plants of the genus Gynostemma.

Origanum Syriacum

According to a specific embodiment, the plants of this species include flavones, monoterpenoids and monoterpenes. Over 60 different compounds have been identified, with the primary ones being carvacrol and thymol ranging to over 80%, while lesser abundant compounds include p-cymene, γ-terpinene, caryophyllene, spathulenol, germacrene-D, β-fenchyl alcohol and δ-terpineol.

The table below shows a profile of the organic compounds identified in Origanum extract through fractional distillation:

Profile of the organic compounds found in the fractions analyzed.

| | | % de Relative Area | | | | | |
|---|---|---|---|---|---|---|---|
| Compound Boiling Point ° C. | Code | F1 | F2 | F3 | F4 | Unoil | Ooil |
| α-thujene 150-152 | MH1 | 5.03 | 0.389 | ND | ND | ND | 1.74 |
| a-pinene 156 | MH2 | 3.01 | ND | ND | ND | ND | 1.07 |
| β-myrcene 166-168 | MH3 | 11.62 | 6.93 | 1.08 | ND | ND | 5.50 |
| Phellandrene 172 | MH4 | 1.32 | 1.00 | ND | ND | ND | 0.72 |
| α-terpinene 174 | MH5 | 8.91 | 8.32 | 2.90 | ND | ND | 5.57 |
| o-cymene 174 | MH6 | 47.96 | 53.97 | 38.14 | 1.31 | 0.973 | 39.13 |
| Limonene 175 | MH7 | 2.29 | 2.71 | 1.25 | ND | ND | 1.58 |
| 1,8-cineole 177 | MO1 | 1.51 | 1.77 | 2.74 | ND | ND | 1.53 |
| γ-terpinene 181-183 | MH8 | 15.59 | 24.43 | 40.57 | 1.40 | 0.94 | 22.34 |
| Thymol 232 | MO2 | ND | ND | ND | 5.08 | 3.77 | 1.71 |
| Carvacrol 237-238 | MO3 | ND | ND | 4.58 | 60.03 | 64.31 | 12.60 |
| Trans-caryophyllene 268 | SeH1 | ND | ND | 2.97 | 18.96 | 13.78 | 3.47 |
| α-humulene 276 | SeH2 | ND | ND | 0.34 | 6.16 | 8.36 | 1.56 |
| Monoterpene hydrocarbons | (MH) | 95.73 | 97.75 | 83.94 | 2.71 | 1.91 | 77.65 |
| Monoterpene oxygenated | (MO) | 1.51 | 1.77 | 7.32 | 65.11 | 68.08 | 15.84 |
| Sesquiterpene hydrocarbons | (SeH) | ND | ND | 3.31 | 25.12 | 22.14 | 5.03 |
| Total identified components | | 97.24 | 99.52 | 94.57 | 92.94 | 92.13 | 98.52 |

Oregano essential oil (Ooil) was obtained through the steam entrainment method and the oil fractions through a fractional distillation system. The first fraction started to distill at a temperature of 82° C. and the last fraction distilling at 140° C., finally undistilled oil (Unoil) was obtained. At the end of the process, five fractions named Fraction 1 (F1), Fraction 2 (F2), Fraction 3 (F3), Fraction 4 (F4), and undistilled oil (Unoil) were obtained.

When Origanum extract was analyzed on HPLC, a variety of phenolic compounds were identified:

Phenolic compounds determined by the HPLC method in O. vulgare ssp. vulgare extract.

| | | Retention Time | UV | MS | Concentration |
|---|---|---|---|---|---|
| Compounds | [M − H]⁻, m/z | (t_{R}), min | Detection | Detection | (mg/g) |
| Gentisic acid | 153 | 3.69 ± 0.04 | NO | YES | <0.02 |
| Chlorogenic | 353 | 6.43 ± 0.05 | YES | YES | 2.10 ± 0.14 |
| acid | | | | | |
| p-Coumaric | 163 | 9.48 ± 0.08 | NO | YES | <0.02 |
| acid | | | | | |
| Hyperoside | 463 | 18.60 ± 0.12 | YES | YES | 1.05 ± 0.03 |
| Isoquercitrin | 463 | 20.29 ± 0.10 | YES | YES | 0.71 ± 0.19 |
| Rutin | 609 | 20.76 ± 0.15 | YES | YES | 0.64 ± 0.15 |
| Rosmarinic | 360 | 21.80 ± 0.10 | YES | YES | 12.83 ± 2.19 |
| acid | | | | | |
| Quercitrin | 447 | 23.64 ± 0.13 | YES | YES | 0.50 ± 0.08 |
| Quercetin | 301 | 27.55 ± 0.15 | NO | YES | <0.02 |
| Luteolin | 285 | 29.64 ± 0.19 | YES | YES | 0.10 ± 0.04 |

Values are the mean ± SD (n = 3).

Total polyphenol content and antioxidant activity of O. vulgare ssp. vulgare extract.

| | TPC (mg | Flavonoid | Caffeic Acids | CUPRAC | FRAP (μM | SO Scavenging |
|---|---|---|---|---|---|---|
| Sample | GAE/g) | (mg RE/g) | (mg CAE/g) | (μM TE/g) | TE/g) | (μM TE/g) |
| O. | 94.69 ± | 38.46 ± | 29.92 ± | 1284 ± | 794.40 ± | 44.00 ± |
| vulgare | 4.03 | 3.54 | 1.08 | 66 | 25.80 | 0.56 |

Each value is the mean ± SD of three independent measurements. TPC, total polyphenols content; SO, superoxide; GAE, gallic acid equivalents; RE, rutin equivalents; CAE, caffeic acid equivalents; TE, Trolox equivalents.

Also contemplated herein are plants of the genus Origanum.

Origanum is a genus of herbaceous perennials and subshrubs in the family Lamiaceae, native to Europe, North Africa, and much of temperate Asia, where they are found in open or mountainous habitats. A few species also naturalized in scattered locations in North America and other regions.

The plants have strongly aromatic leaves and abundant tubular flowers with long-lasting coloured bracts. The genus includes the important group of culinary herbs: marjoram (Origanum majorana) and oregano (Origanum vulgare).

Examples include, but are not limited to:

Origanum acutidens (Hand.-Mazz.) Ietsw.—Turkey, Iraq
Origanum×adanense Baser & H. Duman—Turkey (O. bargyli×O. laevigatum)
Origanum×adonidis Mouterde—Lebanon (O. libanoticum×O. syriacum subsp. bevanii)
Origanum akhdarense Ietsw. & Boulos—Cyrenaica region of eastern Libya
Origanum amanum Post—Hatay region of Turkey
Origanum×barbarae Bornm.—Lebanon (O. ehrenbergii×O. syriacum subsp. bevanii)
Origanum bargyli Mouterde—Turkey, Syria
Origanum bilgeri P. H. Davis—Antalya region of Turkey
Origanum boissieri Ietsw.—Turkey
Origanum calcaratum Juss.—Greece
Origanum compactum Benth.—Spain, Morocco
Origanum cordifolium (Montbret & Aucher ex Benth.) Vogel—Cyprus
Origanum cyrenaicum Beg. & Vacc.—Cyrenaica region of eastern Libya
Origanum dayi Post—Israel
Origanum dictamnus L.— hop marjoram, Cretan dittany, dittany of Crete—endemic to Crete
Origanum×dolichosiphon P. H. Davis—Seyhan region of Turkey (O. amanum×O. laevigatum)
Origanum ehrenbergii Boiss.—Lebanon
Origanum elongatum (Bonnet) Emb. & Maire—Morocco
Origanum floribundum Munby—Algeria
Origanum×haradjanii Rech.f—Turkey (O. laevigatum×O. syriacum subsp. bevanii)
Origanum haussknechtii Boiss.—Turkey
Origanum husnucan-baseri H. Duman, Aytac & A. Duran—Turkey
Origanum hypericifolium O. Schwarz & P. H. Davis—Turkey
Origanum×intercedens Rech.f.—Greece, Turkey (O. onites×O. vulgare subsp. hirtum)
Origanum×intermedium P. H. Davis—Denizli region of Turkey (O. onites×O. sipyleum)
Origanum isthmicum Danin—Sinai
Origanum jordanicum Danin & Kunne—Jordan
Origanum laevigatum Boiss.—Turkey, Syria, Cyprus
Origanum leptocladum Boiss.—Turkey
Origanum libanoticum Boiss.—Lebanon
Origanum majorana L.—(sweet) marjoram—Turkey, Cyprus; naturalized in scattered locations in Europe, North Africa, North+South America
Origanum×lirium Heldr. ex Halacsy—Greece (O. scabrum×O. vulgare subsp. hirtum)
Origanum×majoricum Cambess.—hardy sweet marjoram—Spain including Balearic Islands (O. majorana×O. vulgare subsp. virens)
Origanum microphyllum (Benth.) Vogel—Crete
Origanum×minoanum P. H. Davis—Crete (O. microphyllum×O. vulgare subsp. hirtum)
Origanum minutiflorum O. Schwarz & P. H. Davis—Turkey
Origanum munzurense Kit Tan & Sorger—Turkey
Origanum×nebrodense Tineo ex Lojac—Sicily (O. majorana×O. vulgare subsp. viridulum)
Origanum onites L.—Greece, Turkey, Sicily
Origanum×pabotii Mouterde—Syria (O. bargyli×O. syriacum subsp. bevanii)
Origanum pampaninii (Brullo & Furnari) Ietsw—Cyrenaica region of eastern Libya
Origanum petraeum Danin—Jordan
Origanum punonense Danin—Jordan
Origanum ramonense Danin—Israel
Origanum rotundifolium Boiss.—Turkey, Caucasus
Origanum saccatum P. H. Davis—Turkey
Origanum scabrum Boiss. & Heldr. in P. E. Boissier—Greece
Origanum sipyleum L. —Turkey, Greek Islands
Origanum solymicum P. H. Davis—Antalya region of Turkey
Origanum symes Carlstrom—Islands of the Aegean Sea
Origanum syriacum L.—Turkey, Cyprus, Syria, Lebanon, Jordan, Palestine, Israel, Sinai, Saudi Arabia
Origanum vetteri Briq. & Barbey—Crete
Origanum vogelii Greuter & Burdet—Turkey
Origanum vulgare L.—oregano—Europe, North Africa, temperate Asia (Iran, Siberia, Central Asia, China, etc.); naturalized in parts of North America, New Zealand, Venezuela.

According to a specific embodiment, the active ingredient or combination thereof includes an organic compound component of Origanum extract.

According to a specific embodiment, the active ingredient or combination thereof is selected from the group consisting of α-thujene α-pinene, β-myrcene, Phellandrene, α-terpinene, o-cymene, Limonene, 1,8-cineole, γ-terpinene, Thymol, Carvacrol, Trans-caryophyllene and α-humulene.

According to a specific embodiment, the active ingredient or combination thereof includes a monoterpene hydrocarbon, an oxygenated monoterpene and a sesquiterpene hydrocarbon.

According to a specific embodiment, the active ingredient or combination thereof includes a phenolic compound, e.g., gentisic acid, chlorogenic acid, p-coumaric acid, hyperoside, isoquercitrin, rutin, rosmarinic acid, quercirtin, quercetin and luteolin.

According to a specific embodiment, the active ingredient or combination thereof includes a mineral, e.g., potassium, calcium, magnesium, phosphorus, aluminum, iron, sodium, boron, zinc, cadmium, selenium.

Sesame

Sesame seeds contain thelignans, sesamolin, sesamin, pinoresinol and lariciresinol. Insoluble 11S globulin and soluble 2S albumin, conventionally termed α-globulin and β-globulin, are the two major storage proteins and constitute 80-90% of total seed proteins in sesame. Comparison of amino acid composition indicated that they are substantially less hydrophobic than the known oleosins, and thus should not be aggregated multimers of oleosins. The results of immuno-recognition to sesame proteins reveals that these three polypeptides are unique proteins gathered in oil bodies, accompanying oleosins and triacylglycerols, during the active assembly of the organelles in maturing seeds. The phospholipid, oleic and linoleic acids, chlorophyll and sesamolin, sesamol and γ-tocopherol are found. 10 compounds [2-furfurylthiol, 2-phenylethylthiol, 2-methoxyphenol, 4-hydroxy2, 5-dimethyl-3[2H]-furanone, 2-pentylpyridine, 2-ethyl-3,5-dimethylpyrazine, acetylpyrazine, [E,E]-2,4-decadienal, 2-acetyl-1-pyrroline and 4-vinyl-2-methoxy-phenol] are quantified. On the basis of high OAVs in oil, especially 2-acetyl-1-pyrroline [roasty], 2-furfurylthiol [coffee-like], 2-phenylethylthiol [rubbery] and 4-hydroxy-2,5-dimethyl3 [2H]-furanone [caramel-like] are elucidated as important contributors to the overall roasty, sulphury odour of the crushed sesame material. The structures of novel sesaminol glucosides isolated from sesame seed are determined to be sesaminol 2′-O-β-d-glucopyranoside, sesaminol 2′-O-β-d-glucopyranosyl [1→2]-O-β-dglucopyranoside and sesaminol 2′-O-β-d-glucopyranosyl [1»2]-O-[β-d-glucopyransyl [1»6]]-[β-dglucopyranoside. Also minor sesame lignans such as -(7S,8′R,8R)-acuminatolide piperitol and pinoresinol (as mentioned).

Also contemplated herein are plants of the genus Sesamum.

Examples include, but are not limited to:

Sesamum abbreviatum Merxm.
Sesamum alatum Thonn.
Sesamum angolense Welw.
Sesamum biapiculatum De Wild.
Sesamum calycinum Welw.
Sesamum capense Burm. f.
Sesamum digitaloides Welw. ex Schinz
Sesamum gracile Endl.
Sesamum hopkinsii Suess.
Sesamum indicum L.
Sesamum lamiifolium Engl.
Sesamum latifolium J. B. Gillett
Sesamum lepidotum Schinz
Sesamum macranthum Oliv.
Sesamum marlothii Engl.
Sesamum mombazense De Wild. & T. Durand
Sesamum parviflorum Seidenst.
Sesamum pedalioides Welw. ex Hiern
Sesamum radiatum Schumach. & Thonn.
Sesamum rigidum Peyr.
Sesamum rostratum Hochst.
Sesamum sabulosum A. Chev.
Sesamum schinzianum Asch.
Sesamum somalense Chiov.
Sesamum thonneri De Wild. & T. Durand
Sesamum triphyllum Welw. ex Asch.

Plants that contain Lignan according to some embodiments of the invention include a wide variety of plant foods, including seeds (flax, pumpkin, sunflower, poppy, sesame), whole grains (rye, oats, barley), bran (wheat, oat, rye), beans, fruit (particularly berries), and vegetables (Broccoli and curly kale are rich sources of lignans. Other vegetables such as white and red cabbage, Brussels sprouts, cauliflower, carrots, green and red sweet peppers are also good sources).

Additional plants that contain Sesamin include but are limited to Eleutherococcus senticosus.

Thus, any combination of the above plants is contemplated including 2, 3, 4, 5, 6, 7 of the plants. According to another embodiment, a combination of extracts or fractions including 2, 3, 4, 5, 6, 7 of the different plants.

Examples include, but are not limited to, Nigella sativa, Thymus vulgaris, Origanum syriacum, Thymbra spicata, Satujera thymbra, Sesamum indicum and Rhus coriaria.

Nigella sativa, Thymus capitatus, Origanum syriacum, Thymbra spicata, Satujera thymbra, Sesamum indicum and Rhus coriaria.

Nigella sativa, Thymus capitatus, Thymus vulgaris, Thymbra spicata, Satujera thymbra, Sesamum indicum and Rhus coriaria.

Nigella sativa, Thymus capitatus, Thymus vulgaris, Origanum syriacum, Satujera thymbra, Sesamum indicum and Rhus coriaria.

Nigella sativa, Thymus capitatus, Thymus vulgaris, Origanum syriacum, Thymbra spicata, Sesamum indicum and Rhus coriaria.

Nigella sativa, Thymus capitatus, Thymus vulgaris, Origanum syriacum, Thymbra spicata, Satujera thymbra, and Rhus coriaria.

Nigella sativa, Thymus capitatus, Thymus vulgaris, Origanum syriacum, Thymbra spicata, Satujera thymbra, Sesamum indicum.

Nigella sativa, Origanum syriacum, Thymbra spicata, Satujera thymbra, Sesamum indicum and Rhus coriaria.

Nigella sativa, Thymus capitatus, Thymus vulgaris, Satujera thymbra, Sesamum indicum and Rhus coriaria.

Nigella sativa, Thymus capitatus, Thymus vulgaris, Origanum syriacum, Sesamum indicum and Rhus coriaria.

Nigella sativa, Thymus capitatus, Thymus vulgaris, Origanum syriacum, Thymbra spicata, and Rhus coriaria.

Nigella sativa, Thymus capitatus, Thymus vulgaris, Origanum syriacum, Thymbra spicata, Satujera thymbra.

Nigella sativa, Thymus capitatus.

Nigella sativa, Thymus vulgaris.

Nigella sativa, Origanum syriacum.

Nigella sativa, Thymbra spicata.

Nigella sativa, Satujera thymbra.

Nigella sativa, Sesamum indicum.

Nigella sativa, Rhus coriaria.

Also contemplated are various combinations without Nigella sativa.

According to another embodiment, a combination of active ingredients e.g., thymoquinone, carvacrol, thymol; thymoquinone, carvacrol; thymoquinone, thymol; carvacrol, thymol.

Nigella sativa, Thymus capitatus, Thymus vulgaris.

Nigella sativa, Thymus vulgaris, Origanum syriacum.

Nigella sativa, Origanum syriacum, Thymbra spicata.

Nigella sativa, Thymbra spicata, Satujera thymbra.

Nigella sativa, Satujera thymbra, Sesamum indicum Rhus coriaria.

According to some embodiments the plants and active ingredients thereof are listed in the Table below.

| | |
|---|---|
| Origanum Syricaum | Carvacrol thymol |
| Thymus Capitatus | Carvacrol |
| | p-cymene |
| | y-terpinene |
| | b-caryophyllene |
| Thymus Vulgaris | Thymol |
| Thymbra Spicata | Carvacrol |
| | y-terpinene |
| | p-cymene |
| Satureja Thymbra | y-terpinene |
| | p-cymene |
| | carvacrol |
| | thymol |
| Sumac | Tannin |
| Seasame | Lignans |
| | Seasamolin |
| | Seasamin |
| | Pinoresinol |
| | Lariciresinol |
| Nigella sativa | Thymoquinone |

Other embodiments, which comprise any of the Nigella sativa, Thymus capitatus, Thymus vulgaris, Origanum syriacum, Thymbra spicata, Satujera thymbra, Sesamum indicum, Rhus coriaria, Panax ginseng and Gynostemme pentaphyllum plants or grenera thereof in combinations of 2, 3, 4, 5, 6, 7 and 8 plants are contemplated herein.

Other embodiments of the method, vaccine, pharmaceutical composition, composition or food supplement of the present invention further comprising cannabis or cannabinoids. According to an aspect of the invention there is provided a food supplement, composition or extracts further including “Beduin Tea” comprising

Rose Leaves Micromeria fruticose, Salvia, cymbopgon (Citral,) Aloysia, Verbena officinalis, Origanum majorana, menthe
According to an aspect of the invention there is provided a food supplement, composition or extracts further including “Beduin Tea” comprising
Thyme, sage, cardamom, cinnamon, black tea, habuk, Marmaya.

The plant part, extract thereof, fraction thereof, active ingredient thereof, synthetic analog thereof, mimetic thereof or combination thereof can be used in the treatment of solid and soft tumors and proliferative diseases.

As used herein, the term “solid and soft tumors and proliferative diseases” refers to an abnormal growth of cells/tissue that does contain cysts or liquid. solid and soft tumors and proliferative diseases may be benign (not cancerous), or malignant (cancerous). Different types of solid and soft tumors and proliferative diseases are named for the type of cells that form them. Examples of solid and soft tumors and proliferative diseases are sarcomas, carcinomas, and lymphomas. “Sarcomas” are cancers arising from connective or supporting tissues such as bone or muscle. “Carcinomas” are cancers arising from glandular cells and epithelial cells, which line body tissues. “Lymphomas” are cancers of the lymphoid organs such as the lymph nodes, spleen, and thymus. As these cells occur in most tissues of the body, lymphomas may develop in a wide variety of organs. Exemplary solid and soft tumors and proliferative diseases which are contemplated herein include but are not limited to sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioblastoma multiforme, astrocytoma, medulloblastoma, craniopharyngioma, ependymoma, pinealoma, hemangioblastoma, acoustic neuroma, oligodendroglioma, cutaneous T cell lymphoma (CTCL), melanoma, neuroblastoma, and retinoblastoma.

solid and soft tumors and proliferative diseases can develop in the muscles, bone, lymphatic system, bone marrow and organs of the body. Examples include mesothelioma, sarcomas, lymphomas, sarcomas as well as cancers of the breast, prostate, kidney, ovaries, pancreas, thyroid, and colon.

Additionally, secondary solid and soft tumors and proliferative diseases can erupt as a consequence of treating blood cancers with radiation or chemotherapy. In fact, solid present the second most common type of tumor following treatment in cancer survivors.

The way solid and soft tumors and proliferative diseases are classified plays an important role in understanding the cancer's pathology, determining the most important course of treatment, and evaluating the patient's prognosis.

solid and soft tumors and proliferative diseases are classified using grades based on the abnormalities pathologists identify in tumor cells and how likely the tumor is to spread. Tumorous tissue that appears similar to the organization of normal, healthy cells and tissue and tends to proliferate relatively slowly are called “well-differentiated.” Fast-proliferating tumor cells that look abnormal and are devoid of normal tissue structures are known as “undifferentiated” or “poorly differentiated.” There are four general tumor grades:
Pathologists typically classify tumors with cells that closely resemble normal cells and proliferate slowly as Grade 1 tumors.
Grade 2 tumor cells have more abnormalities in their structure, have moderate cell differentiation and replicate faster than grade 1 tumors.
Tumors classified as either grade 3 or “high grade” have poor cell tissue differentiation and spread more quickly than grade 1 and 2 tumors.
Grade 4 tumors lack cell differentiation altogether and look starkly different from healthy cells and lower grade tumors.

While many cancers are classified using this system, it's important to note that some solid and soft tumors and proliferative diseases types are defined using other grading systems.

For example, doctors may classify breast cancer on mitotic rate, degree of tumor activity in milk ducts (tubule formation), and the size and shape of the nuclei found in tumors cells (known as nuclear grade). Each of these three categories receive a score ranging from 1 to 3. A score of 1 indicates that tumor tissue more closely resembles healthy cells and tissue. A score of “3” indicates is associated with cells and tissue that have the most abnormal appearance. After assigning a score to each of the three categories, the values are then added together for a composite score that ranges from 3 to 9. The values fall into three different tumor classifications:
Low grade or well-differentiated tumors receive a composite score of 3 to 5.
Tumors identified as being intermediate grade or moderately differentiated range from 6 to 7 in scoring.
And tumors receiving scores of 8 or 9 are identified as being poorly differentiated.

The oncology community uses the Gleason scoring system to grade prostate cancer the pathological results of prostate biopsy samples. The pathologist compares the appearance of the diseased tissue to the healthy tissue and assigns a score of 1 to 5 for the tissue. The abnormal tissue that appears most commonly in the tumor(s) is called the primary pattern, while the secondary pattern the next most frequent appearing tissue pattern.

The scores for the primary and secondary patterns are added together for a Gleason score-results of which fall into four categories:
Gleason X means the pathologist could not determine the Gleason score.
Gleason 2-6 is associated with well-differentiated tumorous tissue.
A Gleason 7 score is used to define moderate differentiated tumorous tissue.
Gleason 8-10 scores means that tumor tissue has poor differentiation or is undifferentiated altogether.

In some embodiments the solid and soft tumors and proliferative diseases is a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioblastoma multiforme, astrocytoma, medulloblastoma, craniopharyngioma, ependymoma, pinealoma, hemangioblastoma, acoustic neuroma, oligodendroglioma, cutaneous T cell lymphoma (CTCL), melanoma, neuroblastoma, and retinoblastoma.

In other embodiments, the solid and soft tumors and proliferative diseases is brain cancer, breast cancer, triple negative breast cancer, bladder cancer, bone cancer, colorectal cancer, lung cancer, kidney cancer, liver cancer, stomach cancer, prostate cancer, sarcoma, melanoma, carcinoma, or a lymphoma.

In some embodiments, the solid and soft tumors and proliferative diseases is prostate cancer, breast cancer, colorectal cancer, pancreatic cancer, or a lymphoma.

In some embodiments the solid and soft tumors and proliferative diseases is a lymphoma.

According to some embodiments of the invention the proliferative disease is Fibroids

According to some embodiments of the invention the proliferative disease is Endometriosis

The plant-derived component or components of the present invention can be co-administered with other medications to increase therapeutic bioavailability, boost therapeutic efficacy, and minimize side effects. The plant-derived component or components of the present invention may be administered in a linear or cyclical form, or in any conformation deemed physiologically appropriate as a means of conveying treatment.

Combination Therapy

In treating, preventing, ameliorating, controlling or reducing solid and soft tumors and proliferative diseases growth and metastases, the compounds and/or components of the present invention may be used in conjunction with the following: (1) cancer vaccination strategies, (2) immune-checkpoint modulators such as antagonistic antibodies against immune-checkpoint inhibitors (anti-PD1, anti-PD-L1, anti-CTLA4, anti-Tim3, anti-VISTA, anti-KIR) or agonistic antibodies against immune-accelerators (anti-Lag3, anti-OX40, anti-ICOS, anti-4-1BB, (3) blocking or depleting antibodies against cell surface proteins commonly up-regulated in transformed cells (CEACAM1, Syndecan-2, GRP78), (4) anti-angiogenic therapies (anti-VEGF, anti-VEGFR, VEGFR small molecule inhibitors), (5) anti-lymphangiogenesis (blocking antibodies or inhibitors against VEGF, FDF2, PDGF as well as its respective receptors), (6) standard chemotherapeutic therapies (such as Gemcitabine, Paclitaxel, FOLFORINOX), (7) irradiation therapy, (8) chemokine antagonists (CCR1, CCR4, CCR6, CXCR4, CXCR2, CXCR7 small molecule inhibitors, blocking antibodies, or depleting antibodies), (9) inhibitors targeting common somatic mutations in cancer such as those specifically targeting the following genes (BRAF, KRAS, NRAS, EGFR, CTNNB1, NOTCH1, PIK3CA, PTEN, APC, FLT3, IDH1, IDH2, KIT, TP53, JAK2).

In some embodiments, the chemotherapeutic therapy agent is selected from Abiraterone Acetate, Afatinib, Aldesleukin, Alemtuzumab, Alitretinoin, Altretamine, Amifostine, Aminoglutethimide Anagrelide, Anastrozole, Arsenic Trioxide, Asparaginase, Azacitidine, Azathioprine, Bendamustine, Bevacizumab, Bexarotine, Bicalutamide, Bleomycin, Bortezomib, Busulfan, Capecitabine, Carboplatin, Carmustine, Cetuximab, Chlorambucil, Cisplatin, Cladribine, Crizotinib, Cyclophosphamide, Cytarabine, Dacarbazine, Dactinomycin, Dasatinib, Daunorubicin, Denileukin diftitox, Decitabine, Docetaxel, Dexamethasone, Doxifluridine, Doxorubicin, Epirubicin, Epoetin Alpha, Epothilone, Erlotinib, Estramustine, Etinostat, Etoposide, Everolimus, Exemestane, Filgrastim, Floxuridine, Fludarabine, Fluorouracil, Fluoxymesterone, Flutamide, folate linked alkaloids, Gefitinib, Gemcitabine, Gem tuzumab ozogamicin, GM-CT-01, Goserelin, Hexamethylmelamine, Hydroxyureas, Ibritumomab, Idarubicin, Ifosfamide, Imatinib, Interferon alpha, Interferon beta, Irinotecan, Ixabepilone, Lapatinib, Leucovorin, Leuprolide, Lenalidomide, Letrozole, Lomustine, Mechlorethamine, Megestrol, Melphalan, Mercaptopurine, Methotrexate, Mitomycin, Mitoxantrone, Nelarabine, Nilotinib, Nilutamide, Octreotide, Ofatumumab, Oprelvekin, Oxaliplatin, Paclitaxel, Panitumumab, Pemetrexed, Pentostatin, polysaccharide galectin inhibitors, Procarbazine, Raloxifene, Retinoic acids, Rituximab, Romiplostim, Sargramostim, Sorafenib, Streptozocin, Sunitinib, Tamoxifen, Temsirolimus, Temozolamide, Teniposide, Thalidomide, Thioguanine, Thiotepa, Tioguanine, Topotecan, Toremifene, Tositumomab, Trametinib, Trastuzumab, Tretinoin, Valrubicin, VEGF inhibitors and traps, Vinblastine, Vincristine, Vindesine, Vinorelbine, Vintafolide (EC145), Vorinostat, a salt thereof, and any combination thereof.

In other embodiments the therapeutic antibody is selected from Abagovomab, Alacizumab pegol, Alemtuzumab, Altumomab pentetate (Hybri-ceaker), Amatuximab, Anatumomab mafenatox, anti-PD-1 antibodies, Apolizumab, Arcitumomab (CEA-Scan), Belimumab, Bevacizumab, Bivatuzumab mertansine, Blinatumomab, Brentuximab vedotin, Cantuzumab mertansine, Cantuzumab ravtansine, Capromab pendetide (Prostascint), Catumaxomab (Removab), Cetuximab (Erbitux), Citatuzumab bogatox, Cixutumumab, Clivatuzumab tetraxetan (hPAM4-Cide), Conatumumab, Dalotuzumab, Denosumab, Drozitumab, Edrecolomab (Panorex), Enavatuzumab, Gemtuzumab, Ibritumomab tiuxetan, Ipilimumab (MDX-101), Ofatumumab, Panitumumab, Rituximab, Tositumomab, Trastuzumab, and any combination thereof.

In further embodiments, the chemotherapeutic agent is a radioisotope, a thymidylate synthase inhibitor, a platinum compound, a vinca alkaloid agent, or any combination thereof.

In some embodiments, the compounds and/or components of the present invention may be used in conjunction with an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing anti-inflammatory agent, for example with a compound such as acetaminophen, aspirin, codeine, biological TNF sequestrants, fentanyl, ibuprofen, indomethacin, ketorolac, morphine, naproxen, phenacetin, piroxicam, a steroidal analgesic, sufentanyl, sunlindac, tenidap, and the like.

In some embodiments, the PD-1 and/or PD-L1 inhibitor is selected from the group consisting of durvalumab, atezolizumab, pembrolizumab, nivolumab, AP-106, AP-105, MSB-2311, CBT-501, avelumab, AK-105, 10-102, 10-103, PDR-001, CX-072, SHR-1316, JTX-4014, GNS-1480, recombinant humanized anti-PD1 mAb (Shanghai Junshi Biosciences), REGN-2810, pelareorep, SHR-1210, PD1/PDL1 inhibitor vaccine (THERAVECTYS), BGB-A317, recombinant humanized anti-PD-1 mAb (Bio-Thera Solutions), Probody targeting PD-1 (CytomX), XmAb-20717, FS-118, PSI-001, SN-PDL01, SN-PD07, PD-1 modified TILs (Sangamo Therapeutics), PRS-332, FPT-155, jienuo mAb (Genor Biopharma), TSR-042, REGN-1979, REGN-2810, resminostat, FAZ-053, PD-1/CTLA-4 bispecific antibody (MacroGenics), MGA-012, MGD-013, M-7824, PD-1 based bispecific antibody (Beijing Hanmi Pharmaceutical), AK-112, AK-106, AK-104, AK-103, BI-754091, ENUM-244C8, MCLA-145, MCLA-134, anti-PD1 oncolytic monoclonal antibody (Transgene SA), AGEN-2034, IBI-308, WBP-3155, JNJ-63723283, MEDI-0680, SSI-361, CBT-502, anti-PD-1 bispecific antibody, dual targeting anti-PD-1/LAG-3 mAbs (TESARO), dual targeting anti-PD-1/TIM-3 mAbs (TESARO), PF-06801591, LY-3300054, BCD-100, STI-1110, pembrolizumab biosimilar, nivolumab biosimilar, PD-L1-TGF-beta therapy, KY-1003, STI-1014, GLS-010, AM-0001, GX-P2, KD-033, PD-L1/BCMA bispecific antibody (Immune Pharmaceuticals), PD-1/Ox40 targeting bispecific antibody (Immune Pharmaceuticals), BMS-936559, anti-PD-1/VEGF-A DARPins (Molecular Partners), mDX-400, ALN-PDL, PD-1 inhibitor peptide (Aurigene), siRNA loaded dendritic cell vaccine (Alnylam Pharmaceuticals), GB-226, PD-L1 targeting CAR-TNK-based immunotherapy (TNK Therapeutics/NantKwest), INSIX RA, INDUS-903, AMP-224, anti-CTLA-4/anti-PD-1 bispecific humanized antibody (Akeso Biopharma), B7-H1 vaccine (State Key Laboratory of Cancer Biology/Fourth Military Medical University), and GX-Dl.

In some embodiments, the PD-1 inhibitor is an antibody selected from Nivolumab, Pembrolizumab, and Pidilizumab.

In some embodiments, the immune checkpoint inhibitor is a CTLA-4 inhibitor. A number of CTLA-4 inhibitors are known in the art. In some embodiments, the CTLA-4 inhibitor is an antibody. In some embodiments the CTLA-4 inhibitor antibody is selected from Ipilimumab, Tremelimumab, AGEN1884, and AGEN2041. In some embodiments, the CTLA-4 inhibitor antibody is Ipilimumab. In some embodiments, the CTLA-4 inhibitor antibody is Tremelimumab. In some embodiments, the CTLA-4 inhibitor antibody is AGEN1884. In some embodiments, the CTLA-4 inhibitor antibody is AGEN2041.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings, male or female, at any age or gender, who suffer from the pathology. Preferably, this term encompasses individuals who are at risk to develop the pathology (e.g., above 65 of age, exposed to cigarette smoke, carcinogens, familial susceptibility to solid and soft tumors and proliferative diseases).

The composition of matter comprising the component(s) (a plant species or genus thereof-derived component selected from the group consisting of a plant part, extract thereof, fraction thereof, active ingredient thereof, synthetic analog thereof, mimetic thereof or combination thereof, wherein said component is capable of treating solid and soft tumors and proliferative diseases) of the present invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the composition of matter comprising the components accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intrapulmonary or intraocular injections.

In various exemplary embodiments of the invention, the composition is provided as a pharmaceutical or dietary supplement dosage form suitable for oral administration. Dosage forms suitable for oral administration include tablets, soft capsules, hard capsules, pills, granules, powders, emulsions, suspensions, sprays, syrups and pellets. In various other embodiments of the invention, the composition is provided as a pharmaceutical dosage form suitable for parenteral administration such as liquid formulations for administration as drops or by injection, or as solid or semisolid dosage forms for suppositories.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport polypeptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin polypeptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

In specific embodiments, the components and/or compositions of the invention are provided in form suitable for administration by inhalation or nasal administration.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (composition of matter comprising the components accountable for the biological effect) effective to prevent, alleviate or ameliorate symptoms or progress of a disorder (e.g. solid and soft tumors and proliferative diseases) or prolong the survival of the subject being treated.

Determination of a therapeutically effective amount is well within the capability of those skilled in the art, especially in light of the detailed disclosure provided herein.

For example, any in vivo or in vitro method of evaluating the severity of the solid and soft tumors and proliferative diseases or related symptoms may be employed.

For any preparation used in the methods of the invention, the therapeutically effective amount or dose can be estimated initially from in vitro and cell culture assays.

For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In another embodiment, the invention provides a nutritional or dietary compositions in the form of foods or beverages, which comprise the component(s) described herein. These foods or beverages comprise various exemplary embodiments of the inventive compositions. These foods or beverages can be prepared or provided as cereals, baby foods, healthy foods, or food for specified health uses such as solid food like chocolate or nutritional bars, semisolid food like cream or jam, or gel; and also as beverages. Specific and non-limiting examples of such food or beverage items include refreshing beverages, lactic acid bacteria beverages, drops, candies, chewing gum, chocolate, gummy candy, yoghurts, ice creams, puddings, soft adzuki bean jellies, jellies, cookies and the like.

In yet other embodiments of the present invention components of the compositions are synthetic analogues of the plant products and extracts herein mentioned.

The present teachings further envisage treating with other anti-viral drugs or anti-inflammatory drugs or anti-coagulants as separate treatments or in a co-formulation.

Without being limited to solid and soft tumors and proliferative diseases but for the sake of example, according to a specific embodiment, the antiviral drug is selected from the group consisting of remdesivir, an interferon, ribavirin, adefovir, tenofovir, acyclovir, brivudin, cidofovir, fomivirsen, foscarnet, ganciclovir, penciclovir, amantadine, rimantadine and zanamivir.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells —A Manual of Basic Technique” by Freshney, Wiley-Liss, N. Y. (1994), Third Edition; “Current Protocols in Immunology” Volumes Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Assays for Treatment of Solid and Soft Tumors and Proliferative Diseases

Many cell-based, in-vitro systems for evaluation of solid and soft tumors and proliferative diseases growth and responsiveness to treatment are available, in addition to traditional in-vivo animal models. To model solid and soft tumors and proliferative diseases, primary as well as cell lines of tumors from a variety of tissues are cultured and then exposed to the therapeutic compositions and/or components. Cell responses, and in particular proliferation, senescence and metabolic activity are determined in the presence or absence of the added compositions and/or components of the invention, in order to evaluate the ability of the compositions and/or components of the invention to reduce or otherwise alter the tumor phenotype. Cells can be propagated in 2-D or 3-D cultures.

Exemplary cell types for in-vitro modeling of solid and soft tumors and proliferative diseases of nearly all organs and tissue are widely commercially available, for example, NCI-H295R cells for adrenal tumors, HT-1376, J82, T24P cells for bladder tumors, DBTRG, LN-18, SF-295, SF-767 and SNB-19 cells for brain tumors, Ca Ski, He La and KB cells for cervical tumors, COLO 205, DLD-1, HCT, LoVo and NCI-H508 cells for colon cancer, HEKn cells for epithelial tumors, OE33 cells for esophageal tumors, A4573 cells for Ewings sarcoma, NHDF and Hs 895T cells for fibroblast-derived tumors, GIST-T1 and NCI-N87 cells for gastric tumors, CAL 27 cells for head and neck tumors, Hep, Hepa and BLN cells for liver tumors, Calu-6, NCI-H596, NCI-H125-Luc, HCC827, LL and LL/2 cells for lung tumors, YAC-1, DB, GRANTA-519, EBC-1, Daudi, Raji and RL cells for lymphoma, HCC70, MCF-7, MDA-MB, SK-BR3 and MX-1 cells for breast tumors, SK-MEL and OCM cells for melanoma, AB1 cells for mesothelioma, RPMI 8226 and OPM-2 cells for myeloma, SK—N—F1 for neuroblastoma, OVCAR cells for ovarian cancer, PANC-1 and Capan cells for pancreatic cancer, PC-3 and VCaP cells for prostate cancer, ACHN and Renca cells for renal cancer, MG-63, A-673 and SW 872 cells for sarcomas, TT and MB-1 cells for Thyroid tumors and SK-LMS cells for vulvar cancer.

Tumor cells can be evaluated in vitro, and some can be used for xenograft growth assays by introduction into animals. In one exemplary embodiment, tumor cells from primary cultures or cell lines are cultured in-vitro, and either injected into the circulation, subcutaneously or directly into the target organ of a mouse or rat, and establishment of tumors, and their growth, can be monitored by direct measurement or detection of labeled cells. Animal hosts can be immune competent or immune deficient (SCID, nude). The anti-tumor efficacy of the compositions and/or components of the invention can be assessed and evaluated at multiple stages of the tumor cell's growth—by administration at the in-vitro cell growth stage (pre-graft), at the stage of introduction into the host animal, and also for effect on established xenograft tumors after they have been allowed to reach a certain size in the host animal.

Animal Models of Solid and Soft Tumors and Proliferative Diseases Growth

Animal models for solid and soft tumors and proliferative diseases include induced animal models, transgenic models and naturally occurring animal models of the hyperproliferative diseases and conditions.

Aside from the animal models mentioned hereinabove, genetically engineered mouse and rat cancers provide powerful in-vivo models of tumors that allow opportunity to evaluate drug delivery, therapeutic response and biomarker expression of tumors in their natural environment. Genetically engineered animal models suitable for assessing efficacy of the compositions and components of the invention include, but are not limited to: MMTV-PyMT mouse mammary tumor genetically engineered mice, K14-HPV16 mice for squamous skin tumors and KB1P breast cancer mouse model.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### Example 2

Treatment of Squamous Cell Carcinoma (SCC) and Basal Cell Sarcoma (BCC)

Squamous cell carcinoma (SCC) of the skin is the second most common form of skin cancer, characterized by abnormal, accelerated growth of squamous cells. When caught early, most SCCs are curable. Squamous cells are flat cells located near the surface of the skin that shed continuously as new ones form. SCCs can appear as scaly red patches, open sores, rough, thickened or wart-like skin, or raised growths with a central depression. At times, SCCs may crust over, itch or bleed. The lesions most commonly arise in sun-exposed areas of the body. Basal cell carcinoma (BCC) is the most common form of skin cancer and the most frequently occurring form of all cancers. BCCs arise from abnormal, uncontrolled growth of basal cells, One of three main types of cells in the top layer of the skin. BCCs can look like open sores, red patches, pink growths, shiny bumps, scars or growths with slightly elevated, rolled edges and/or a central indentation. At times, BCCs may ooze, crust, itch or bleed. The lesions commonly arise in sun-exposed areas of the body. Due to their slow growth, most BCCs are curable and cause minimal damage when caught and treated early. Both SCC and BCC most often occurs when DNA damage from exposure to ultraviolet (UV) radiation from the sun (see skincancer.org).

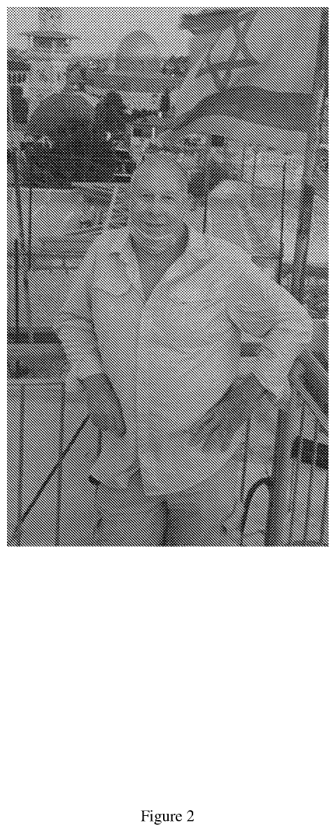

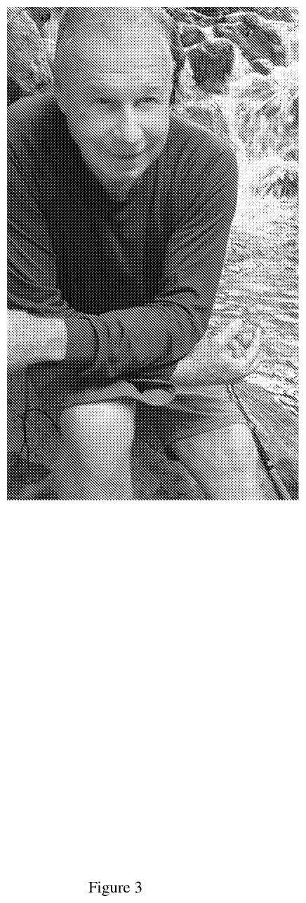

A patient with history of BCC was diagnosed with SCC located on the left ear after 3 positive biopsies. A suspected involvement of BCC as well was noted but with no lymph nodes involvement. Beside the tumor on the left ear, a discoloration and pigmentation of the face were evident. The skin cancer patient was diagnosed before the current treatment with a BCC and an intrusive SSC
After the anti cancer treatment of the present invention t was found in both ultrasound and CT examination—that all tissues were clear and that no lymph nodes were involved After three months of treatment with compositions of the present invention herein. described the discoloration and pigmentation of the face were eradicated.as shown in FIG. 2 and FIG. 3.
Reference is herein made to the method, vaccine, pharmaceutical composition, composition or food supplement of the present invention herein described, wherein said solid and soft tumors and proliferative diseases is selected from the group consisting of sarcomas and carcinomas such as Fibrosarcoma, Myxosarcoma, Liposarcoma, Chondrosarcoma, Osteogenic Sarcoma, Chordoma, Angiosarcoma, Endotheliosarcoma, Lymphangiosarcoma, Lymphangioendotheliosarcoma, Synovioma, Mesothelioma, Ewing's Tumor, Leiomyosarcoma, Rhabdomyosarcoma, Colon Carcinoma, Pancreatic Cancer, Breast Cancer, Ovarian Cancer, Prostate Cancer, Squamous Cell Carcinoma, Basal Cell Carcinoma, Adenocarcinoma, Sweat Gland Carcinoma, Sebaceous Gland Carcinoma, Papillary Carcinoma, Papillary Adenocarcinomas, Cystadenocarcinoma, Medullary Carcinoma, Bronchogenic Carcinoma, Renal Cell Carcinoma, Hepatocellular Carcinoma, Bile Duct Carcinoma, Choriocarcinoma, Seminoma, Embryonal Carcinoma, Wilm's Tumor, Cervical Cancer, Testicular Tumor, Lung Carcinoma, Small Cell Lung Carcinoma, Bladder Carcinoma, Epithelial Carcinoma, Glioblastoma Multiforme, Astrocytoma, Medulloblastoma, Craniopharyngioma, Ependymoma, Pinealoma, Hemangioblastoma, Acoustic Neuroma, Oligodendroglioma, Cutaneous T Cell Lymphoma (CTCL), Cutaneous B Cell Lymphoma (CBCL), Melanoma, Neuroblastoma, Retinoblastoma, Hodgkin's Lymphoma, Non-Hodgkin's Lymphoma, Diffuse Large B Cell Lymphoma, Chronic Lymphatic Leukemia, Mantle Cell Lymphoma, Follicular Lymphoma, Splenic Marginal Zone Lymphoma, Nodal Marginal Zone Lymphoma, Extranodal Marginal Zone Lymphoma, Burkitt's Lymphoma, Plasmablastic Lymphoma, Peripheral Tcell Lymphoma NOS, Hairy Cell Leukemia (HCL), Acute Lymphocytic Leukemia (ALL), Acute Myeloid Leukemia (AML), Acute Promyelocytic Leukemia (APL), Chronic Lymphocytic Leukemia (CLL), Chronic Myeloid Leukemia (CML), Myeloproliferative Neoplasms (MPN) And Systemic Mastocytosis, Papillary thyroid cancer, Noninvasive Follicular Thyroid Neoplasm, Follicular Thyroid, cancer, Medullary Thyroid Cancer, Anaplastic Thyroid Cancer, Thyroid Lymphoma, Squamous Cell Thyroid Carcinoma, Thyroid Sarcoma, Hürthle Cell Carcinoma

### Example 3

A woman suffering from colon cancer was treated with compositions of the present invention. CT and Ultra Sound examination showed reduction and in some places, disappearance of tumours after 13 days of treatment with compositions of the present invention.

The invention is not intended to be limited to the embodiment illustrated and described above, but it can be modified and varied within the scope and spirit of the invention as defined by the following claims

### APPENDIX

Comparative chemical composition of the essential oil of Thymus vulgaris L. from different geographical sources
A. RAAL¹, E. ARAK¹, A. ORAV²

### ¹Institute of Pharmacy, University of Tartu

### Nooruse St. 1, 50411 Tartu, Estonia

### ²Institute of Chemistry, Tallinn University of Technology

### Ehitajate tee 5, 19086 Tallinn, Estonia

### SUMMARY

Variations in the essential oil composition of Thymus vulgaris L. cultivated in Estonia and in other European countries were determined using capillary gas chromatographic analysis methods. Fifty-nine components were identified, representing over 95% of the total oil yield. The principal components in the oils of common thyme were thymol (0.9%-75.7%), carvacrol (1.5%-83.5%), p-cymene (4.3%-34.4%), γ-terpinene (0.9%-19.7%), linalool (0.4%-4.8%), (E)-β-caryophyllene (0.5%-9.3%) and terpinen-4-ol (tr.-3.8%). The sum of phenolic compounds (thymol and carvacrol) in the oils studied varied from 19.4% to 84.4%, and the sum of their precursors (p-cymene and γ-terpinene) ranged from 5.796 to 38.5%. Thymol content was predominant in the oils of Holland (65.5%) and of Estonia (75.7%) but carvacrol content predominated in the Greek thyme oil (83.5%). Armenian thyme oil contained only 17.0% of thymol, but it was rich in neral and citronellol (32.5%), borneol (4.3%)citronellal (4.0%), 1,8-cineol (4.0%) and methyl eugenol and thymol acetate (7.5%). In Estonia, the thymol, thymol-carvacrol and thymol-p-cymene-γ-terpinene chemotypes of the common thyme are distinguishable.
Key words: Thymus vulgaris L., Labiatae, common thyme, essential oil, different geographical sources, thymol, carvacrol, p-cymene, γ-terpinene

Within the genus Thymus there are many species and subspecies. Most of them, including Thymus vulgaris L., contain thymol and carvacrol as the main components, whereas the variations occur in the concentrations of 1,8-cineole, camphor, citral, carvone, monoterpene alcohols, as well as acetates and sesquiterpene alcohols [1-14]. These chemotypes, especially rich in phenolic terpenoids, showed strong antioxidant activities [15, 16]. Only two Thymus species are known in Estonia. Common thyme (Thymus vulgaris L.) is cultivated and wild thyme (Thymus serpyllum L.) grows wild. A study of essential oil composition of wild thyme origi-nating from various natural places of growth in Estonia showed the presence of at least three chemotypes [17]. Contrary to the literature data concerning other countries, thymol and carvacrol were not the main components of the Estonian wild thyme oil.

In the present work we determined the composition of the essential oil, using commercial common thyme samples from different European countries and samples cultivated in Estonia. The differences in the contents of the biologically active constituents were studied. Concentrations of the main thyme oil constituents from Estonia were compared to samples of other European countries.

### Materials and Methods

Plant materials (commercial Thymi herba) were obtained from retail pharmacies of various European countries in 2000 (France), 2001 (Hungary, Holland), 2002 (Russia, Greece, Estonia), and 2003 (Scotland, Moldavia, Armenia), The Estonian samples were gathered in summers of 2001, 2002 and 2003 from different places of growth in Estonia. Voucher specimens have been deposited at the Institute of Pharmacy, University of Tartu, Estonia.

### Capillary Gas Chromatography

The essential oil was isolated from dried herb of common thyme by the distillation method described in the European Pharmacopoeia [18]. The oils were analysed using a Chrom-5 chromatograph with FID on two fused silica capillary columns (50 m×0.20 mm i.d.) with nonpolar polydimethylsiloxane (NB-30) and polar polyethylene glycol 20M (NB-20M) stationary phases (Nordion, Finland). Film thickness of both stationary phases was 0.25 μm. Helium was used as a carrier gas, with split rate 1:150 and the flow rate 20-25 cm/sec. The temperature programme was from 50-250° C. at 2° C./min, the injector temperature was 250° C. A 3390A Hewlett-Packard integrator was used for data processing.

### Gas Chromatography/Mass Spectrometry

The GC-MS data were obtained on a Hewlett-Packard 5988A instrument. The MS conditions were as follows: El mode 70 eV, ion source temperature 200° C. GC conditions were 60-280° C. at 5° C./min with an internal hold time of 2 minutes. Helium was used as a carrier gas at a flow rate of 20 cm/sec. A fused silica capillary column AT-5, poly(5%-phenyl-95%-dimethylsiloxane), was used (25 m×0.25 mm i.d., film thickness 0.25 μm). The injector temperature was 280° C.

### Identification and Quantitative Evaluation

Compounds were identified by comparing the retention indices (RI) of the GC peaks on NB-30 and NB-20M columns with the RI values of standard compounds, our RI data bank and the literature 119-211. The results obtained were confirmed by GC-MS. The quantitative composition of the oils was calculated on the basis of the GC peak areas on the NB-30 column without FID response factor correction, using the normalisation method.

### Results and Discussion

The RI values of essential oil components of Thymus vulgaris L. on two columns of different polarity, the percentage composition of the thyme oils from Estonia and other European countries are presented in Table 1.

TABLE 1
Composition of the essential oil from Thymus vulgaris
L. of different origins, %.

| | retention index | | |
|---|---|---|---|
| compound | NB-30 | NB-20M | content, % |
| tricyclene | 920 | 1010 | 0-0.1 |
| α-thujene | 924 | 1021 | 0-1.7 |
| α-pinene | 931 | 1019 | 0-1.5 |
| camphene | 945 | 1063 | 0-1.9 |
| sabinene | 967 | 1118 | 0-0.7 |
| 1-octen-3-ol | 968 | 1454 | 0-1.3 |
| β-pinene | 971 | 1115 | tr-1.1 |
| myrcene | 984 | 1162 | tr-5.1 |
| α-phellandrene | 998 | 1167 | 0-0.3 |
| 3-carene | 1005 | 1148 | 0-0.2 |
| α-terpinene | 1011 | 1180 | tr-1.4 |
| p-cymene | 1015 | 1270 | 4.3-34.4 |
| 1,8-cineole | 1022 | 1205 | 0.1-4.0 |
| limonene | 1024 | 1195 | tr-7.9 |
| (Z)-β-ocimene | 1028 | 1232 | tr-0.2 |
| (E)-β-ocimene | 1040 | 1250 | 0-0.3 |
| γ-terpinene | 1050 | 1240 | 0.9-19.7 |
| cis-linalool oxide* | 1056 | 1420 | 0-1.0 |
| trans-sabinene hydrate | 1058 | 1466 | 0-0.6 |
| trans-linalool oxide | 1076 | 1455 | 0-0.2 |
| terpinolene | 1081 | 1276 | 0-0.4 |
| linalool | 1089 | 1551 | 0.4-4.8 |
| camphor | 1123 | 1513 | 0-3.8 |
| citronellal | 1143 | 1480 | 0-4.0 |
| isoborneol* | 1152 | | 0-3.0 |
| borneol | 1154 | 1720 | 0-4.3 |
| p-cymen-8-ol* | 1162 | 1860 | 0-0.4 |
| terpinen-4-ol | 1166 | 1602 | 0-3.8 |
| α-terpineol | 1177 | 1713 | 0-1.5 |
| (Z)-dihydrocarvone | 1181 | | 0-0.5 |
| thymol methyl ether | 1218 | 1580 | tr-3.3 |
| neral and citronellol | 1220 | 1677 | 0-32.5 |
| | 1222 | 1800 | |
| carvone | 1224 | 1735 | 0-3.7 |
| carvacrol methyl ether | 1230 | 1584 | 0-2.2 |
| geraniol | 1243 | 1855 | 0-5.8 |
| geranial | 1264 | 1725 | 0-1.5 |
| (E)-anethole and isobornyl acetate* | 1264 | 1837 | 0-1.1 |
| | 1262 | | |
| bornyl acetate | 1273 | 1574 | 0-2.4 |
| thymol | 1280 | 2197 | 0.9-75.7 |
| carvacrol | 1290 | 2210 | 1.5-83.5 |
| methyl eugenol* | 1332 | 1920 | 0-7.5 |
| thymol acetate* | 1334 | | |
| α-terpinyl acetate | 1335 | 1700 | 0-0.4 |
| carvacryl acetate* | 1347 | | 0-0.9 |
| neryl acetate | 1353 | 1724 | 0-0.3 |
| α-copaene | 1371 | 1485 | 0-0.6 |
| β-bourbonene | 1380 | 1510 | 0-0.3 |
| (E)-β-caryophyllene | 1418 | 1589 | 0.5-9.3 |
| α-ionone | 1426 | | 0-0.5 |
| bicyclosesquiphellandrene* | 1436 | | 0-0.3 |
| α-humulene | 1449 | 1658 | 0-0.8 |
| alloaromadendrene | 1457 | 1632 | 0-0.5 |
| γ-muurolene | 1472 | 1690 | 0-0.8 |
| germacrene D | 1478 | 1700 | 0-4.3 |
| α-muurolene | 1494 | 1720 | 0-0.6 |
| bicyclogermacrene* | 1490 | 1722 | 0-0.8 |
| β-bisabolene | 1500 | 1736 | 0-2.6 |
| γ-cadinene | 1505 | 1744 | 0-0.5 |
| δ-cadinene | 1517 | 1746 | 0-1.0 |
| hedycaryol* | 1530 | 2077 | 0-0.6 |
| selina-3,7(11)-diene* | 1540 | | 0-2.4 |
| germacrene-B* | 1555 | | 0-1.0 |
| spathylenol | 1570 | 2124 | 0-1.0 |
| caryophyllene oxide | 1575 | 1980 | 0.1-2.5 |
| γ-eudesmol* | 1612 | | 0-0.2 |
| T-cadinol | 1630 | 2170 | 0-0.5 |
| α-cadinol | 1646 | 2217 | 0-0.4 |
| farnesol* | 1659 | | 0-0.7 |
| component groups: | | | |
| aliphatic compounds | | | tr-1.3 |
| monoterpenes | | | 8.3-42.1 |
| (p-cymene + γ-terpinene) | | | 5.7-38.5 |
| oxygenated monoterpenes | | | 40.4-86.8 |
| (thymol + carvacrol) | | | 19.4-84.4 |
| sesquiterpenes | | | 0.3-17.6 |
| oxygenated sesquiterpenes | | | 0.1-4.5 |
| total, % | | | 96.0-99.8 |

The components identified in the highest yields are printed in bold; tr—traces (<0.05%),
*tentatively identified.

Fifty-nine components were identified in the samples studied, representing over 95% of the total oil. The main compound group in the oils was oxygenated monoterpenoids (40.4%-86.8%), including phenols (thymol and carvacrol): 19.4%-84.4%. Monoterpenes constituted 8.3%-42.1% of the oils, including phenolic precursors (p-cymene and γ-terpinene): 5.7%-38.5%. Sesquiterpenes made up 0.3%-17.6% of the thyme oils. The major sesquiterpenes in the oils were (E)-β-caryophyllene (0.5%-9.3%), germacrene D (0%-4.3%), β-bisabolene (0%-2.6%) and selina-3,7(11)-diene (0%-2.4%). The other sesquiterpenes made up less than 1% in all the samples. From the oxygenated sesquiterpenes identified in the thyme oils only caryophyllene oxide (0.1%-2.5%) was found to form over 1%.

A comparison of thyme oil composition from samples of different geographical sources showed some variability of the majority of biologically active constituents. In the oils of Greek origin, carvacrol amounted to 83.596. In other samples studied, this value varied from 2.2% to 4.1%. In the case of two thyme samples from Estonia and Holland the oil contained more thymol (75.7%, 67.5% and 65.5%, respectively) than the other samples (0.9-49.0%). The sum of concentrations of precursors of phenols, p-cymene and γ-terpinene, varied from 5.7% to 38.5%, and these values were lowest in the oils from Armenia (5.7%) and Greece (7.8%). The total concentration of four major constituents (thymol, carvacrol, p-cymene and γ-terpinene) in the thyme oils studied ranged from 67.7% to 92.2%. The only exception was the oil from Armenia, where this value formed only 25.1%. The Armenian thyme oil was rich in neral and citronellol (32.5%), methyl eugenol and thymol acetate (7.5%), borneol (4.3%), citronellal (4.0%) and 1,8-cineol (4.0%).

As shown in Table 2, the thymol chemotype is clearly distinguishable in the Estonian samples 6 and 7 (content of thymol 75.7% and 67.5%, respectively). Samples 4, 8 and 10 were rich in thymol (22.5%-45.1%) and carvacrol (29.9%-34.6%), while samples 1, 2, 3 and 5 were rich in thymol (41.7%-49.0%) and p-cymene (14.6%-22.2%). Unlike the other oils studied, sample 9 contained relatively little thymol, carvacrol and p-cymene (total 45.6%), but it was rich in monoterpenes (myrcene—5.1%) and sesquiterpenes (β-caryophyllene—9.3%, germacrene D—4.3%).

The results of this work have established noticeable quantitative differences in the case of biologically active compounds in common thyme oils from different geographical sources. Consequently the pharmacological effects of these medicinal plants, being of a basically antimicrobial and antibacterial nature, are also likely to differ.

The oil from Holland and two oils from Estonia belong to the thymol chemotype, while the oils from France, Hungary, Russia and Scotland belong to the thy-mol-p-cymene rich chemotype. Only in Estonia, the thymol—carvacrol and thy-mol-p-cymene-γ-terpinene chemotypes are distinguishable. The oil from Greece was found to be of a carvacrol-rich chemotype. Unlike the other oils, the oil from Armenia contained high quantities of neral and citronellol.

TABLE 2
Concentration of the main essential oil components
of Thymus vulgaris L. from different geographical sources.

| tested | concentration, % | | | | | | |
|---|---|---|---|---|---|---|---|
| samples | myrcene | p-cymene | γ-terpinene | linalool | terpinen-4-ol | thymol | caracrol |
| France | 0.8 | 28.1 | 4.5 | 2.4 | tr. | 45.7 | 3.8 |
| Hungary | 0.3 | 25.5 | 1.1 | 1.9 | 0.9 | 45.6 | 4.1 |
| Holland | 0.4 | 10.0 | 1.9 | 2.0 | 0.8 | 65.5 | 2.8 |
| Russia | 0.7 | 22.5 | 3.9 | 2.9 | 1.1 | 48.0 | 3.4 |
| Greece | 0.8 | 6.6 | 1.2 | 0.8 | 0.5 | 0.9 | 83.5 |
| Scotland | 0.5 | 34.4 | 4.1 | 4.8 | 2.3 | 31.5 | 3.5 |
| Moldavia | tr. | 16.4 | 0.9 | 1.8 | 3.8 | 47.8 | 2.6 |
| Armenia | 0.3 | 4.6 | 1.1 | 0.4 | 2.2 | 17.0 | 2.4 |
| Estonia: | | | | | | | |
| sample 1 | 1.7 | 22.2 | 10.9 | 2.1 | tr. | 49.0 | 2.2 |
| sample 2 | 1.7 | 20.2 | 9.1 | 2.3 | 0.2 | 49.0 | 2.9 |
| sample 3 | 2.5 | 14.6 | 19.7 | 1.8 | 0.2 | 47.2 | 1.9 |
| sample 4 | 0.4 | 6.5 | 3.4 | 2.0 | 0.9 | 45.1 | 29.9 |
| sample 5 | 0.7 | 16.9 | 9.2 | 2.9 | 0.7 | 41.7 | 10.1 |
| sample 6 | 0.4 | 4.3 | 3.8 | 2.1 | 0.8 | 75.7 | 4.1 |
| sample 7 | 1.2 | 11.6 | 6.2 | 2.1 | 0.2 | 67.5 | 2.8 |
| sample 8 | 0.7 | 16.4 | 4.9 | 1.7 | 0.3 | 28.5 | 34.6 |
| sample 9 | 5.1 | 7.9 | 7.0 | 2.7 | 0.4 | 29.2 | 1.5 |
| sample 10 | 1.4 | 17.7 | 9.6 | 2.2 | 1.1 | 22.5 | 32.1 |
| sample 11 | 1.6 | 6.2 | 4.4 | 0.7 | 0.6 | 39.4 | 6.0 |

### CONCLUSIONS

The principal components in the essential oils of common thyme from different geographical sources are thymol, carvacrol, p-cymene, γ-terpinene, linalool, (E)-β-caryophyllene and terpinen-4-ol.

In Estonia, the thymol, thymol-carvacrol and thymol-p-cymene-γ-terpinene chemotypes of the common thyme are distinguishable.

### ACKNOWLEDGEMENT

Financial support for the work reported here was provided by the Estonian Science Foundation (grant No. 4332).

### REFERENCES

1. Weiss B, Flück H. Studies on the variability of content and composition of volatile oil in leaf- and herb-drugs of Thymus vulgaris L. Pharmaceut Acta Helv 1970; 45:169-83.
2. Granger R, Passet J. Thymus vulgaris spontane de France: Races chimiques et chemotaxonomie. Phytochem 1973; 12:1683-91.
3. Oszagyan M, Simandi B, Sawinsky, Kery A, Lemberkovics E, Fekete J. Supercritical fluid extraction of volatile compounds from lavandin and thyme. Flay Fragr J 1996; 11:157-65.
4. Venskutonis R, Poll L, Larsen M. Influence of drying and irradiation on the composition of volatile compounds of Thyme (Thymus vulgaris L.). Flay Fragr J 1996; 11:123-8.
5. Blum C, Kubeczka K R, Becker K. Supercritical fluid chromatography & mass chromatography of thyme extracts (Thymus vulgaris L.). J Chromatogr 1997; A 773:377-80.
6. Bhaskara Reddy M V, Angers P, Gosselin A, Arul J. Characterization and use of essential oil from Thymus vulgaris against Botrutis cinerea and Rhizopus stolonifer in strawberry fruits. Phytochem 1998; 47:1515-20.
7. Guillen M D, Manzanos M J. Study of the composition of the different parts of a Spanish Thymus vulgaris L. plant. Food Chem 1998; 63:373-83.
8. Rosch P, Popp J, Kiefer W. Raman and structure enhanced Raman spectroscopic investigation of Lamiaceae plants. J Molec Struc 1999; 121-124:480-1.
9. Bicchi C, Cordero C, lori C, Rubiolo P, Sandra P. Headspace sorptive extraction (HSSE) in the headspace analysis of aromatic and medicinal plants. J High Resol Chromatogr 2000; 23:539-46.
10. Bicchi C, Drigo S, Rubiolo P. Influence of fibre coating in headspace solid-phase microextraction-gas chromatographic analysis of aromatic and medicinal plants. J Chromatogr 2000; A 892:469-85.
11. Pothier J, Galand N, El Ouali M, Viel C. Comparison of planar chromatographic methods (TLC, OPLC, AMD) applied to essential oils of wild thyme and seven chemotypes of thyme. II Farmaco 2001; 56:505-11.
12. Hubaib H, Speroni E, Di Pietra A M, Cavrini V. GC/MS evaluation of Thyme (Thymus vulgaris L.) oil composition and variations during the vegetative cycle. J Pharmac Biomed Analysis 2002; 29:691-700.
13. Thompson J D, Chalchat J C, Michet A, Linhart Y B, Ehlers B. Qualitative and quantitative variation in monoterpene co-occurrence and composition in the essential oil of Thymus vulgaris chemotypes. J Chem Ecol 2003; 29:858-80.
14. Lucchesi M E, Chemat F, Smadja J. Solvent free microwave extraction of essential oil from aromatic herbs: comparison with conventional hydrodistillation. J Chromatogr 2004; A 1043:323-7.
15. Piccaglia R, Marotti M. Characterization of several aromatic plants grown in northern Italy. Flay Fragr J 1993; 8:115-22.
16. Dorman H J D, Deans S G, Noble R S, Surai P. Evaluation in vitro plant essential oils as natural antioxidants. J Essent Oil Res 1995; 7:645-51.
17 Raal A, Paaver U, Arak E, Orav A. Content and composition of the Thymus serpyllum L. growing wild in Estonia. Medicina (Kaunas) 2004; 40:795-800.
18. European Pharmacopoeia 4^{th} Editon. Strasbourg: EDQM, 1999:2545-7.
19. Zenkevich I G. Analytical parameters of component of essential oils for their GC and GC-MS identification. Mono-and sesquiterpenes. Rastit Resur 1996; 32:48-58.
20. Zenkevich I G. Analytical parameters of component of essential oils for their GC and GC-MS identification. Oxygen containing derivatives of mono-and sesquiterpenes hydrocarbons. Rastit Resur 1997; 33:16-27.
21. Zenkevich I G. Analytical parameters of essential oil's components for their GC and GC-MS identification. Acetates of terpenic alcohols. Rastit Resur 1999; 35:30-7.
   PORÓWNANIE SKLADU CHEMICZNEGO OLEJKU Z TYMIANKU POSPOLITEGO (Thymus vulgaris L.) Z RÓŻNYCH REJÓNOW

A. RAAL¹, E. ARAK¹, A. ORAV²

¹Instytut Farmacji, Uniwersytet Tartu

Nooruse St. 1, 50411 Tartu, Estonia

²Instytut Chemii, Politechnika Tallińska

Ehitajate tee 5, 19086 Tallin, Estonia

Streszczenie

Różnice skladu chemicznego olejku uzyskanego z tymianku pospolitego (Thymus vulgaris L.) uprawianego w Estonii i innych krajach europejskich określono za pomocą metody kapilarnej chromatografii gazowej. Określono 59 skladników, tworzących w sumie ponad 95% skladu olejku. Glównymi skladnikami olejków uzyskiwanych z tymianku pospolitego byly tymol (0.9%-75.7%), karwakrol (1.59%-83.5%), p-cymen (4.3%-34.4%), γ-terpinen (0.9%-19.7%), linalol (0.4%-4.8%), (E)-β-kariofylen (0.5%-9.3%) oraz terpinen-4-ol (od ilośi śladowych do 3.8%). Lączna ilość związków fenolowych (tymolu i karwakrolu) w badanych olejkach wynosila od 19.4% do 84.4%, a lączna ilość ich prekursorów (p-cymenu i γ-terpinenu)—od 5.7% do 38.5%. Zawartość tymolu byla najwyisża w olejkach uzyskiwanych z tymianku pochodzącego z Holandii (65.5%) i Estonii (75.7%), natomiast w olejku uzyski-wanym z roślin pochodzących z Grecji dominowal karwakrol (83.5%). Olejek pozyskiwany z tymianku rosnącego w Armenii zawieral tylko 17.0% tymolu, charakteryzowal siȩ natomiast wysoką zawartośią neralu i citronelolu (32.5%), borneolu (4.3%), citronelalu (4.0%), 1,8-cineolu (4.0%) oraz metyloeugenolu i octanu tymolu (7.5%). W wypadku tymianku pospolitego rosnącego w Estonii można wyróżnić chemotypy tymolu, tymolu-karwakrolu oraz tymolu-p-cymenu-γ-terpinenu.

Slowa kluczowe: Thymus vulgaris L., Labiatae, tymianek pospolity, olejek, różne źródla geograficzne, tymol, karwakrol, p-cymen, γ-terpinen

1. A method of preventing or treating solid and soft tumors and proliferative diseases in a subject in need thereof, the method comprising administering to the subject an effective amount of the pharmaceutical composition of claim 3.

2. A vaccine against solid and soft tumors and proliferative diseases comprising an effective amount of a plant species or genus thereof-derived component selected from a plant part, extract thereof, fraction thereof, active ingredient thereof, synthetic analog thereof, mimetic thereof or combination thereof, wherein said component is capable of treating solid and soft tumors and proliferative diseases and wherein said plant species is selected from Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

3. A pharmaceutical composition comprising an effective amount of a plant species or genus thereof-derived component selected from a plant part, extract thereof, fraction thereof, active ingredient thereof, synthetic analog thereof, mimetic thereof or combination thereof, wherein said component is capable of treating solid and soft tumors and proliferative diseases and wherein said plant species is selected from Nigella sativa, Thymus capitatus, Thymus vulgaris, Origanum syriacum, Thymbra spicata, Satujera thymbra, Sesamum indicum, Rhus coriaria, Gynostemma pentaphyllum, Boswellia sacra and Panax ginseng.

4. A composition of matter comprising at least 2 of a plant species or genus thereof-derived components selected from a plant part, extract thereof, fraction thereof, active ingredient thereof, synthetic analog thereof, mimetic thereof or combination thereof, wherein said component is capable of treating solid and soft tumors and proliferative diseases and wherein said plant species is selected from Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

5. A food supplement comprising a combination of at least 2 of a plant species or genus thereof-derived component selected from a plant part, extract thereof, fraction thereof, active ingredient thereof, synthetic analog thereof, mimetic thereof or combination thereof, wherein said component is capable of treating solid and soft tumors and proliferative diseases and wherein said plant species is selected from Nigella sativa, Thymus capitatus, Thymus vulgaris, Origanum syriacum, Thymbra spicata, Satujera thymbra, Sesamum indicum Rhus coriaria, Gynostemma pentaphyllum, Boswellia sacra and Panax ginseng.

6. The method of claim 1, wherein said solid and soft tumors and proliferative diseases is selected from Fibrosarcoma, Myxosarcoma, Liposarcoma, Chondrosarcoma, Osteogenic Sarcoma, Chordoma, Angiosarcoma, Endotheliosarcoma, Lymphangiosarcoma, Lymphangioendotheliosarcoma, Synovioma, Mesothelioma, Ewing's Tumor, Leiomyosarcoma, Rhabdomyosarcoma, Colon Carcinoma, Pancreatic Cancer, Breast Cancer, Ovarian Cancer, Prostate Cancer, Squamous Cell Carcinoma, Basal Cell Carcinoma, Adenocarcinoma, Sweat Gland Carcinoma, Sebaceous Gland Carcinoma, Papillary Carcinoma, Papillary Adenocarcinomas, Cystadenocarcinoma, Medullary Carcinoma, Bronchogenic Carcinoma, Renal Cell Carcinoma, Hepatocellular Carcinoma, Bile Duct Carcinoma, Choriocarcinoma, Seminoma, Embryonal Carcinoma, Wilm's Tumor, Cervical Cancer, Testicular Tumor, Lung Carcinoma, Small Cell Lung Carcinoma, Bladder Carcinoma, Epithelial Carcinoma, Glioblastoma Multiforme, Astrocytoma, Medulloblastoma, Craniopharyngioma, Ependymoma, Pinealoma, Hemangioblastoma, Acoustic Neuroma, Oligodendroglioma, Cutaneous T Cell Lymphoma (CTCL), Cutaneous B Cell Lymphoma (CBCL), Melanoma, Neuroblastoma, Retinoblastoma, Hodgkin's Lymphoma, Non-Hodgkin's Lymphoma, Diffuse Large B Cell Lymphoma, Chronic Lymphatic Leukemia, Mantle Cell Lymphoma, Follicular Lymphoma, Splenic Marginal Zone Lymphoma, Nodal Marginal Zone Lymphoma, Extranodal Marginal Zone Lymphoma, Burkitt's Lymphoma, Plasmablastic Lymphoma, Peripheral Tcell Lymphoma NOS, Hairy Cell Leukemia (HCL), Acute Lymphocytic Leukemia (ALL), Acute Myeloid Leukemia (AML), Acute Promyelocytic Leukemia (APL), Chronic Lymphocytic Leukemia (CLL), Chronic Myeloid Leukemia (CML), Myeloproliferative Neoplasms (MPN) And Systemic Mastocytosis, Papillary thyroid cancer, Noninvasive Follicular Thyroid Neoplasm, Follicular Thyroid, cancer, Medullary Thyroid Cancer, Anaplastic Thyroid Cancer, Thyroid Lymphoma, Squamous Cell Thyroid Carcinoma, Thyroid Sarcoma, and Hürthle Cell Carcinoma.

7. The method of claim 1, wherein said solid and soft tumors and proliferative diseases is brain cancer, breast cancer, triple negative breast cancer, bladder cancer, bone cancer, colorectal cancer, lung cancer, kidney cancer, liver cancer, stomach cancer, prostate cancer, sarcoma, melanoma, carcinoma, or a lymphoma, fibroids, or endometriosis.

8. The method of claim 1, wherein the solid and soft tumors and proliferative diseases is a lymphoma.

9. The method of claim 1, wherein said component comprises at least 2 components.

10. The method of claim 1, wherein said component comprises at least 3 components.

11. The method of claim 1, wherein said component comprises at least 4 components.

12. The method of claim 1, wherein said component comprises at least 5 components.

13. The method of claim 1, wherein said component comprises 5-10 components.

14. The method of claim 1, wherein said component comprises thymoquinone or an analog thereof.

15. The method of claim 1, wherein said component comprises thymol or an analog thereof.

16. The method of claim 1, wherein said component comprises carvacrol or an analog thereof.

17. The method of claim 1, wherein said component comprises bromelain or an analog thereof.

18. The method of claim 1, wherein the plant species or genus thereof-derived component further comprises extracts of pineapple comprising bromelain or an analog thereof.

19. The method of claim 1, wherein the pharmaceutical composition further comprises cannabis or cannabinoids.

20. The method of claim 1, wherein the pharmaceutical composition further comprises Tryptophan.

21. The method of claim 1, wherein the plant species or genus thereof-derived component—further comprises “Beduin Tea” comprising Rose Leaves Micromeria fruticose, Salvia, cymbopgon (Citral,) Aloysia, Verbena officinalis, Origanum majorana, menthe.

22. The method of claim 1—wherein the plant species or genus thereof-derived component further comprises “Beduin Tea” comprising Thyme, sage, cardamom, cinnamon, black tea, habuk, Marmaya.
